(19) United States
(12) Patent Application Publication
(10) Pub. No.: US 2026/0207493 A1
(43) Pub. Date: Jul. 23, 2026

(54) FORMULATIONS OF ANTI-PD1 ANTIBODIES

(71) Applicant: Formycon AG, Martinsried/Planegg (DE)

(72) Inventors: Rainer Sigl, Landsberg Am Lech (DE); Felix Steffen Hintermeier, Martinsried/Planegg (DE)

(73) Assignee: Formycon AG, Martinsried/Planegg (DE)

(21) Appl. No.: 19/139,093

(22) PCT Filed: Dec. 21, 2023
(86) PCT No.: PCT/EP2023/087186

§ 371 Date: Jun. 13, 2025

(30) Foreign Application Priority Data
Dec. 21, 2022 (EP) 22215254.8

(51) Int. Cl. A61K 9/08; A61K 9/00; A61K 47/12; A61K 47/18; A61K 47/26; C07K 16/28

(52) U.S. Cl. CPC A61K 9/08; A61K 9/0019; A61K 47/12; A61K 47/183; A61K 47/26; C07K 16/2818; C07K 2317/24; C07K 2317/76; C07K 2317/94

(57) ABSTRACT

The present invention relates to pharmaceutical compositions of an anti-PD1 antibody comprising a mono- or dicarboxylic buffer, arginine and/or lysine and a surfactant.

### FIELD OF THE INVENTION

The present invention relates to pharmaceutical compositions of an anti-PD1 antibody comprising a mono- or dicarboxylic buffer, arginine and/or lysine and a surfactant.

### BACKGROUND OF THE INVENTION

Programmed cell death protein 1 (PD-1) is a cell surface protein which acts as an immune checkpoint and prevents autoimmune diseases by suppressing T cell inflammatory activity.

PD-1 binds two ligands, PD-L1 and PD-L2. PD-L1 overexpression has been found in several cancer types and it was found that the inhibition of the interaction between PD-1 and PD-L1 can enhance T cell responses and thereby mediate antitumor activity.

Several antibodies targeting either PD-1 or PD-L1 have been developed. Pembrolizumab, marketed under the trade name Keytruda®, is a humanized IgG4 antibody which binds to PD-1 and blocks its interaction with PD-L1. It is presently authorized for the treatment of several cancer types including melanoma, non-small cell lung cancer, head and neck squamous cell carcinoma, bladder cancer and Hodgkin's lymphoma. Nivolumab, marketed under the trade name Opdivo®, is a fully human IgG4 antibody which binds to PD-1 and blocks its interaction with PD-L1. It is presently authorized for the treatment of melanoma, non-small cell lung cancer and renal cell carcinoma.

WO 2012/135408 and WO 2019/160751 A2 disclose liquid and lyophilized formulations of pembrolizumab comprising a histidine buffer, polysorbate 80 and sucrose.

WO 2017/054646 A1 discloses formulations of an anti-PD1 antibody containing sodium acetate, α,α-trehalose dihydrate and polysorbate 20, pH 5.2.

WO 2018/028383 A1 describes a pharmaceutical formulation comprising an anti-PD1 antibody, citrate, histidine, mannitol, sodium chloride, edetate and polysorbate 20 or polysorbate 80, pH 5.5 to 6.5.

WO 2018/187057 A1 discloses pharmaceutical compositions comprising an anti-PD1 antibody, histidine, sucrose, proline and polysorbate 80, pH 6.0.

WO 2018/204368 A1 describes formulations of an anti-PD1 antibody comprising a buffer, a stabilizer, a non-ionic surfactant and an anti-oxidant.

WO 2019/142149 A2 discloses pharmaceutical compositions comprising an anti-PD1 antibody, histidine, sucrose, polysorbate 20 and EDTA, pH 6.5.

WO 2019/171253 A1 describes a pharmaceutical composition comprising anti-PD1 antibody, a disaccharide, a buffer, a chelating agent and a polysorbate, pH 4.5 to 5.5.

WO 2020/097141 A1 describes a pharmaceutical composition comprising anti-PD1 antibody, a buffer, a stabilizer, a surfactant and an anti-oxidant.

WO 2021/118321 A1 discloses a pharmaceutical preparation of an anti-PD1 antibody which is free of buffer.

EP 2 471 554 A1 discloses formulations of a biopharmaceutical drug comprising at least one mono- or dicarboxylic acid or a salt thereof.

WO 2021/123202 describes a liquid pharmaceutical composition comprising anti-PD1 antibody, a histidine or a citrate buffer, a sugar or sugar alcohol and a non-ionic surfactant.

WO 2022/268887 describes a liquid pharmaceutical composition comprising anti-PD1 antibody, a citrate buffer, an amino acid such as arginine or lysine and a non-ionic surfactant.

Nevertheless, there is still a need for stable formulations of anti-PD1 antibodies which are safe for administration to human patients, in particular for subcutaneous administration.

### SUMMARY OF THE INVENTION

The present invention relates to a liquid pharmaceutical composition comprising:
(a) an anti-human PD1 antibody;
(b) arginine and/or lysine;
(c) a non-ionic surfactant; and
(d) a mono- or dicarboxylic acid with a backbone of 2-6 carbon atoms or at least one salt thereof, preferably an unbranched mono- or dicarboxylic acid with a backbone of 4, 5 or 6 carbon atoms,
wherein the pH of the composition is between 5.0 and 5.8.

In one embodiment, the mono- or dicarboxylic acid with a backbone of 2-6 carbon atoms or at least one salt thereof is selected from the group consisting of acetate, adipate, succinate, malate, glutamate and tartrate.

In one embodiment, the buffer is present in a concentration of 1 mM to 50 mM, preferably of 10 mM.

In one embodiment, the non-ionic surfactant is polysorbate 20 or polysorbate 80, preferably is polysorbate 80.

In one embodiment, the non-ionic surfactant is present in a concentration of 0.1 mg/ml to 0.4 mg/ml, preferably of 0.2 mg/ml.

In one embodiment, the liquid pharmaceutical composition comprises arginine in a concentration of 50 mM to 350 mM, preferably of 150 mM.

In one embodiment, the liquid pharmaceutical composition comprises lysine in a concentration of 50 mM to 150 mM, preferably of 70 mM to 140 mM or 70 mM to 130 mM.

In one embodiment, the pharmaceutical composition does not comprise trehalose, preferably does not comprise any sugar or sugar alcohol.

In one embodiment, the pharmaceutical composition does not comprise methionine or histidine, preferably does not comprise any antioxidant.

In one embodiment, the anti-human PD1 antibody is pembrolizumab.

In one embodiment, the anti-human PD1 antibody is present in a concentration of 100 to 200 mg/ml, preferably of 165 mg/ml or in a concentration of 20 to 80 mg/ml, preferably of 25 mg/ml.

The present invention also relates to a liquid pharmaceutical composition consisting of acetate buffer, arginine, polysorbate 20 or polysorbate 80, pembrolizumab and water for injection and having a pH of 5.0 to 5.8.

The present invention also relates to a liquid pharmaceutical composition consisting of adipate buffer, arginine or lysine, polysorbate 20 or polysorbate 80, optionally trehalose, pembrolizumab and water for injection and having a pH of 5.0 to 5.8.

The present invention also relates to a liquid pharmaceutical composition consisting of succinate buffer, arginine, polysorbate 20 or polysorbate 80, pembrolizumab and water for injection and having a pH of 5.0 to 5.8.

The present invention also relates to a liquid pharmaceutical composition consisting of acetate buffer, lysine, polysorbate 20 or polysorbate 80, pembrolizumab and water for injection and having a pH of 5.0 to 5.8.

The present invention also relates to a liquid pharmaceutical composition consisting of adipate buffer, arginine or lysine, polysorbate 20 or polysorbate 80, pembrolizumab and water for injection and having a pH of 5.0 to 5.8.

The present invention also relates to a liquid pharmaceutical composition consisting of succinate buffer, lysine, polysorbate 20 or polysorbate 80, pembrolizumab and water for injection and having a pH of 5.0 to 5.8.

The present invention also relates to said liquid pharmaceutical composition for use in the treatment of cancer, preferably wherein the cancer is melanoma, non-small cell lung carcinoma, classical Hodgkin lymphoma, urothelial carcinoma, head and neck squamous cell carcinoma, renal cell carcinoma, colorectal cancer, small cell lung cancer, microsatellite instability-high or mismatch repair deficient cancer, primary mediastinal large B-cell lymphoma, gastric or gastroesophageal junction adenocarcinoma, oesophageal carcinoma, hepatocellular carcinoma, Merkel cell carcinoma, endometrial carcinoma, tumor mutational burden-high cancer, cutaneous squamous cell carcinoma, triple-negative breast cancer or cervical cancer.

### DETAILED DESCRIPTION OF THE INVENTION

The present invention as illustratively described in the following may suitably be practiced in the absence of any element or elements, limitation or limitations, not specifically disclosed herein.

The present invention will be described with respect to particular embodiments, but the invention is not limited thereto, but only by the claims.

Where the term “comprising” is used in the present description and claims, it does not exclude other elements. For the purposes of the present invention, the term “consisting of” is considered to be a preferred embodiment of the term “comprising” and excludes the presence of other elements. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. a cell or organism is defined to be obtainable by a specific method, this is also to be understood to disclose a cell or organism which is obtained by this method.

Where an indefinite or definite article is used when referring to a singular noun, e.g. “a”, “an” or “the”, this includes a plural of that noun unless something else is specifically stated.

The term “pharmaceutical composition” as used herein refers to any composition comprising a chemical or biological substance or active ingredient which composition is intended for use in the medical cure, treatment, or prevention of disease and which is in such a form as to permit the active ingredient to be effective. In particular, a pharmaceutical composition does not contain excipients which are unacceptably toxic to a subject to which the composition is to be administered. The pharmaceutical compositions are sterile, i.e. aseptic and free from all living microorganisms and their spores. The pharmaceutical composition used in the present invention is liquid and stable.

In a “liquid composition” the pharmaceutically active agent, e.g. the anti-PD1 antibody, can be combined with a variety of excipients to ensure a stable active medication following storage. In one embodiment, the liquid pharmaceutical composition used in the invention is at no point lyophilized, i.e. the production method does not contain a lyophilization step and the composition is not lyophilized for storage. Liquid compositions can be stored in vials, IV bags, ampoules, cartridges, and prefilled or ready-to-use syringes.

In another embodiment the liquid composition is lyophilized after its preparation. The terms “lyophilization”, “lyophilized” and “freeze-dried” refer to a process in which the material to be dried is first frozen and then the ice or frozen solvent is removed by sublimation in a vacuum environment. Preferably, the lyophilized formulation is prepared by lyophilizing the liquid pharmaceutical composition of the present invention. The skilled person is aware of protocols for lyophilization.

A “stable” liquid composition is one in which the anti-PD1 antibody contained therein essentially retains its physical stability and/or chemical stability and/or biological activity upon storage for a certain period. Preferably, the composition essentially retains upon storage its physical and chemical stability, as well as its biological activity. Various analytical techniques for measuring protein stability are available in the art and are reviewed, for example, in Peptide and Protein Drug Delivery, 247-301, Vincent Lee Ed, Marcel Dekker, Inc, New York, New York, Pubs (1991) and Jones, Adv Drug Delivery Rev, 1993, 10:29-90. For example, stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection), by assessing charge heterogeneity using cation exchange chromatography or capillary zone electrophoresis, amino-terminal or carboxy-terminal sequence analysis, mass spectrometric analysis, SDS-PAGE analysis to detect aggregated or fragmented molecules, peptide map (for example tryptic or LYS-C) analysis, evaluating biological activity or binding of the antagonist, etc. Suitable methods for determining stability are described in the examples herein.

Preferably, the pharmaceutical composition is stable at a temperature of about 40° C. for at least 1 to 2 weeks, and/or is stable at a temperature of about 5° C. for at least 3 months, preferably 6 months or 9 months and more preferably one year, and/or is stable at a temperature of about 25° C. for at least two weeks or one month. Furthermore, the formulation is preferably stable following freezing (to, e.g., −80° C.) and thawing of the formulation at 25° C. as described in the examples herein, for example following 1, 2, 3 or 4 cycles of freezing and thawing.

For example, in the pharmaceutical composition of the present invention the percentage of high molecular weight species of the anti-PD1 antibody relative to the total amount of the anti-PD1 antibody as measured by size exclusion chromatography is not more than 10%, preferably not more than 5%, more preferably not more than 3% and most preferably not more than 2% after storage for two weeks at a temperature of about 40° C.

Alternatively, in the pharmaceutical composition of the present invention the percentage of high molecular weight species of the anti-PD1 antibody relative to the total amount of the anti-PD1 antibody as measured by size exclusion chromatography is not more than 10%, preferably not more than 5%, more preferably not more than 3% and most preferably not more than 2% after storage for one month, three months, six months or nine months at a temperature of about 5° C.

A “buffer” is an aqueous solution consisting of a mixture of a weak acid and its conjugate base or vice versa which resists changes in its pH and therefore keeps the pH at a nearly constant value. The buffer of the present invention preferably has a pH in the range from about 5.0 to about 5.8, preferably from about 5.2 to about 5.7, more preferably from about 5.2 to 5.5 and most preferably has a pH of about 5.2 or 5.5.

The buffer used in the present invention is based on a mono- or dicarboxylic acid with a backbone of 2 to 6 carbon atoms and/or at least one salt thereof, preferably an unbranched mono- or dicarboxylic acid with a backbone of 4, 5 or 6 carbon atoms. A monocarboxylic acid comprises one carboxylic group and a dicarboxylic acid comprises two carboxylic groups. As used herein, the term “mono- or dicarboxylic acid with a backbone of n carbon atoms” refers to mono- or dicarboxylic acids which have a straight alkyl- or alkylene backbone with n carbon atoms. Said backbone may have side chains such as methyl groups, but the carbon atoms comprised in these side chains do not count as carbon atoms of the backbone.

According to this definition, cyclic sugar acids such as ascorbic acid do not qualify as “mono- or dicarboxylic acid with a backbone of n carbon atoms”, as they do not have a straight alkyl- or alkylene backbone. The term “2 to 6 carbon atoms” as used herein is intended to include compounds having a backbone of 2, 3, 4, 5 or 6 carbon atoms.

In one embodiment, the buffer is an acetate buffer. The acetate buffer may be prepared by mixing acetic acid with a acetate salt such as sodium acetate or by mixing acetic acid with a base such as sodium hydroxide, arginine and/or lysine. The acetate buffer has a concentration of 1 mM to 50 mM, preferably of 2 mM to 40 mM, more preferably of 3 mM to 30 mM, even more preferably of 4 mM to 20 mM and most preferably of 10 mM.

According to the invention the acetate buffer has a pH in the range from about 5.0 to 5.8, preferably from about 5.1 to 5.7, more preferably from about 5.2 to 5.5 and most preferably has a pH of about 5.2 or 5.5.

In one embodiment, the acetate buffer has a concentration of 10 mM and a pH of 5.5. In one embodiment, the acetate buffer has a concentration of 10 mM and a pH of 5.2.

In one embodiment, the buffer is an adipate buffer. The adipate buffer may be prepared by mixing adipic acid with a adipate salt such as sodium adipate or by mixing adipic acid with a base such as sodium hydroxide, arginine and/or lysine. The adipate buffer has a concentration of 1 mM to 50 mM, preferably of 2 mM to 40 mM, more preferably of 3 mM to 30 mM, even more preferably of 4 mM to 20 mM and most preferably of 10 mM.

According to the invention the adipate buffer has a pH in the range from about 5.0 to 5.8, preferably from about 5.1 to 5.7, more preferably from about 5.2 to 5.5 and most preferably has a pH of about 5.2 or 5.5.

In one embodiment, the adipate buffer has a concentration of 10 mM and a pH of 5.5. In one embodiment, the adipate buffer has a concentration of 10 mM and a pH of 5.2.

In one embodiment, the buffer is a succinate buffer. The succinate buffer may be prepared by mixing succinic acid with a succinate salt such as sodium succinate or by mixing succinic acid with a base such as sodium hydroxide, arginine and/or lysine. The succinate buffer has a concentration of 1 mM to 50 mM, preferably of 2 mM to 40 mM, more preferably of 3 mM to 30 mM, even more preferably of 4 mM to 20 mM and most preferably of 10 mM.

According to the invention the succinate buffer has a pH in the range from about 5.0 to 5.8, preferably from about 5.1 to 5.7, more preferably from about 5.2 to 5.5 and most preferably has a pH of about 5.2 or 5.5.

In one embodiment, the succinate buffer has a concentration of 10 mM and a pH of 5.5. In one embodiment, the succinate buffer has a concentration of 10 mM and a pH of 5.2.

In one embodiment, the buffer is a malate buffer. The malate buffer may be prepared by mixing malic acid with a malate salt such as sodium malate or by mixing malic acid with a base such as sodium hydroxide, arginine and/or lysine. The malate buffer has a concentration of 1 mM to 50 mM, preferably of 2 mM to 40 mM, more preferably of 3 mM to 30 mM, even more preferably of 4 mM to 20 mM and most preferably of 10 mM.

According to the invention the malate buffer has a pH in the range from about 5.0 to 5.8, preferably from about 5.1 to 5.7, more preferably of about 5.2 to 5.5 and most preferably has a pH of about 5.2 or 5.5.

In one embodiment, the malate buffer has a concentration of 10 mM and a pH of 5.5. In one embodiment, the malate buffer has a concentration of 10 mM and a pH of 5.2.

A “surfactant” as used herein refers to an amphiphilic compound, i.e. a compound containing both hydrophobic groups and hydrophilic groups which lowers the surface tension (or interfacial tension) between two liquids or between a liquid and a solid. A “non-ionic surfactant” has no charged groups in its head. The formation of insoluble particles during freeze/thaw cycles of antibody-containing compositions can be remarkably inhibited by addition of surfactants. Examples of “non-ionic surfactants” include e.g. polyoxyethylene glycol alkyl ethers, such as octaethylene glycol monododecyl ether, pentaethylene glycol monododecyl ether; polyoxypropylene glycol alkyl ethers; glucoside alkyl ethers, such as decyl glucoside, lauryl glucoside, octyl glucoside; polyoxyethylene glycol octylphenol ethers, such as triton X-100; polyoxyethylene glycol alkylphenol ethers, such as nonoxynol-9; glycerol alkyl esters, such as glyceryl laurate; polyoxyethylene glycol sorbitan alkyl esters, such as polysorbate; sorbitan alkyl esters, such as spans; cocamide MEA, cocamide DEA, dodecyldimethylamine oxide; block copolymers of polyethylene glycol and polypropylene glycol, such as poloxamers (e.g. poloxamer 188); and polyethoxylated tallow amine (POEA).

The liquid pharmaceutical compositions of the present invention can contain one or more of these surfactants in combination. In a preferred embodiment the pharmaceutical compositions of the present invention contain only one non-ionic surfactant.

Preferred non-ionic surfactants for use in the liquid pharmaceutical compositions of the present invention are polysorbates such as polysorbate 20, 40, 60 or 80, and more preferably polysorbate 20 (i.e. Tween 20) or polysorbate 80 (i.e. Tween 80). In another embodiment, the non-ionic surfactant used in the liquid pharmaceutical compositions of the present invention is poloxamer 188.

The concentration of the non-ionic surfactant is in the range of 0.005 to 0.06% (w/v), preferably in the range of 0.008 to 0.05% (w/v), and most preferably in the range of 0.01 to 0.04% (w/v), relative to the total volume of the composition.

In a preferred embodiment, the non-ionic surfactant is polysorbate 20. In a preferred embodiment, the non-ionic surfactant is polysorbate 20 with a concentration in the range of 0.05 to 0.6 mg/ml, preferably in the range of 0.08 to 0.5 mg/ml, and most preferably in the range of 0.1 to 0.4 mg/ml. In a preferred embodiment, the non-ionic surfactant is polysorbate 20 with a concentration of 0.2 mg/ml.

In another preferred embodiment, the non-ionic surfactant is polysorbate 80 with a concentration in the range of 0.05 to 0.6 mg/ml, preferably in the range of 0.08 to 0.5 mg/ml, more preferably in the range of 0.1 to 0.4 mg/ml and most preferably of 0.2 mg/ml.

In a particularly preferred embodiment, the non-ionic surfactant is polysorbate 80 with a concentration of 0.2 mg/ml.

In one embodiment, the liquid pharmaceutical composition of the present invention does not comprise a sugar or sugar alcohol. Sugar alcohols are organic compounds derived from a sugar which contain a hydroxyl group attached to each carbon atom. Suitable sugar alcohols include glycerol, mannitol, sorbitol, and xylitol. In one embodiment, the liquid pharmaceutical composition of the present invention does not comprise a non-reducing sugar. In one embodiment, the liquid pharmaceutical composition of the present invention does not comprise sucrose. In one embodiment, the liquid pharmaceutical composition of the present invention does not comprise trehalose.

The liquid pharmaceutical composition of the present invention may comprise one or more amino acids other than histidine. Amino acids are organic compounds that contain amino and carboxyl groups and a side chain which is specific for each amino acid. Amino acids which can be present in the liquid pharmaceutical composition of the present invention may be selected from the group consisting of arginine, lysine, aspartic acid, glutamic acid, serine, threonine, asparagine, glutamine, cysteine, glycine, proline, alanine, valine, isoleucine, methionine, phenylalanine, tyrosine and tryptophan.

In one embodiment, the liquid pharmaceutical composition of the present invention comprises arginine. In one embodiment, the liquid pharmaceutical composition of the present invention comprises lysine. In one embodiment, the liquid pharmaceutical composition of the present invention comprises arginine and lysine. In one embodiment, arginine is the only amino acid present in the liquid pharmaceutical composition of the present invention. In one embodiment, lysine is the only amino acid present in the liquid pharmaceutical composition of the present invention. In one embodiment, arginine and lysine are the only amino acids present in the liquid pharmaceutical composition of the present invention.

The arginine and/or lysine may be added to the liquid pharmaceutical composition as the free base or as the hydrochloride salt thereof. In one embodiment, the arginine is added as a mixture of the free base arginine with the hydrochloride salt of arginine, i.e. arginine hydrochloride (arginine-HCl). In one embodiment, the lysine is added as a mixture of the free base lysine with the hydrochloride salt of lysine, i.e. lysine hydrochloride (lysine-HCl). In one embodiment, the arginine is added as a mixture of the free base arginine with the hydrochloride salt of arginine, i.e. arginine hydrochloride, and the lysine is added as a mixture of the free base lysine with the hydrochloride salt of lysine, i.e. lysine hydrochloride.

In one embodiment, the liquid pharmaceutical composition of the present invention comprises arginine in a concentration of 50 mM to 350 mM, preferably of 60 mM to 330 mM or 60 mM to 300 mM or 60 mM to 280 mM, more preferably of 70 mM to 250 mM or 70 mM to 220 mM or 70 mM to 200 mM and most preferably of 70 mM or 150 mM. In one embodiment, the liquid pharmaceutical composition of the present invention comprises arginine in a concentration of 150 mM. In one embodiment, the liquid pharmaceutical composition of the present invention comprises arginine in a concentration of 70 mM.

In one embodiment, the liquid pharmaceutical composition of the present invention comprises lysine in a concentration of 50 mM to 200 mM, preferably of 55 mM to 180 mM or 55 mM to 170 mM, more preferably of 60 mM to 160 mM or 60 mM to 150 mM, even more preferably of 70 mM to 140 mM and most preferably of 70 mM or 120 mM or 130 mM or 140 mM. In one embodiment, the liquid pharmaceutical composition of the present invention comprises lysine in a concentration of 70 mM. In one embodiment, the liquid pharmaceutical composition of the present invention comprises lysine in a concentration of 120 mM. In one embodiment, the liquid pharmaceutical composition of the present invention comprises lysine in a concentration of 130 mM. In one embodiment, the liquid pharmaceutical composition of the present invention comprises lysine in a concentration of 140 mM.

In one embodiment, the liquid pharmaceutical composition of the present invention does not contain sodium chloride. In one embodiment, the liquid pharmaceutical composition of the present invention does not contain any sodium salt. In one embodiment, the liquid pharmaceutical composition of the present invention does not contain any inorganic salt. As used herein, an “inorganic salt” refers to an ionic compound which has osmoregulatory properties. An inorganic salt such as sodium chloride (NaCl) can dissociate in solution into its constituent ions, i.e. NaCl dissociates into Na+ and Cl− ions, which both affect the osmotic pressure, i.e. the osmolality, of the solution. Exemplary inorganic salts which are not present in the liquid pharmaceutical composition of the present invention are potassium chloride, calcium chloride, sodium chloride, sodium phosphate, potassium phosphate and sodium bicarbonate.

In one embodiment, the liquid pharmaceutical composition of the present invention does not contain EDTA. In one embodiment, the liquid pharmaceutical composition of the present invention does not contain pentetic acid. In one embodiment, the liquid pharmaceutical composition of the present invention does not contain EDTA and pentetic acid. In one embodiment, the liquid pharmaceutical composition of the present invention does not contain any chelating agent. Chelating agents can form at least one bond with a metal atom. A chelating agent is typically a multidentate ligand that can be used in compositions as a stabilizer to complex with species, which might otherwise promote instability. Exemplary chelating agents include aminopolycarboxylic acids, hydroxyaminocarboxylic acids, N-substituted glycines, 2-(2-amino-2-oxocthyl) aminoethane sulfonic acid (BES), deferoxamine (DEF), niacinamide, desoxycholates, ethylenediaminetetraacetic acid (EDTA), diethylenetriaminepentaacetic acid (DTPA), nitrilotriacetic acid (NTA), N-2-acetamido-2-iminodiacetic acid (ADA), bis(am inoethyl)glycolether, N,N,N′,N′-tetraacetic acid (EGTA), trans-diaminocyclohexane tetraacetic acid (DCTA), N-hydroxyethyliminodiacetic acid (HIMDA), N,N-bis-hydroxyethylglycine (bicine), N-(trishydroxymethylmethyl) glycine (tricine), glycylglycine, sodium desoxycholate, ethylenediamine; propylenediamine; diethylenetriamine; triethylenetetraamine (trien), ethylenediaminetetraaceto EDTA; disodium EDTA, EDTA, calcium EDTA oxalic acid and malate.

In one embodiment, the liquid pharmaceutical composition of the present invention does not contain methionine. In one embodiment, the liquid pharmaceutical composition of the present invention does not contain L-methionine or L-methionine-HCl. In one embodiment, the liquid pharmaceutical composition of the present invention does not contain any anti-oxidant. Anti-oxidants are compounds that inhibit oxidation by reacting with oxidizing agents. In the present invention arginine is not considered as anti-oxidant. In the present invention lysine is not considered as anti-oxidant. In the present invention arginine and lysine are not considered as anti-oxidants.

In one embodiment, the liquid pharmaceutical composition of the present invention does not contain proline. In one embodiment, the liquid pharmaceutical composition of the present invention does not contain glycine. In one embodiment, the liquid pharmaceutical composition of the present invention does not contain glutamic acid. In one embodiment, the liquid pharmaceutical composition of the present invention does not contain serine. In one embodiment, the liquid pharmaceutical composition of the present invention does not contain tyrosine. In one embodiment, the liquid pharmaceutical composition of the present invention does not contain tryptophan. In one embodiment, the liquid pharmaceutical composition of the present invention does not contain leucine. In one embodiment, the liquid pharmaceutical composition of the present invention does not contain phenylalanine. In one embodiment, the liquid pharmaceutical composition of the present invention does not contain threonine. In one embodiment, the liquid pharmaceutical composition of the present invention does not contain aspartate. In one embodiment, the liquid pharmaceutical composition of the present invention does not contain asparagine. In one embodiment, the liquid pharmaceutical composition of the present invention does not contain glutamine. In one embodiment, the liquid pharmaceutical composition of the present invention does not contain alanine. In one embodiment, the liquid pharmaceutical composition of the present invention does not contain cysteine. In one embodiment, the liquid pharmaceutical composition of the present invention does not contain isoleucine. In one embodiment, the liquid pharmaceutical composition of the present invention does not contain valine. In one embodiment, the liquid pharmaceutical composition of the present invention does not contain any amino acid in addition to arginine.

In one embodiment, the liquid pharmaceutical composition of the present invention does not contain any amino acid in addition to lysine. In one embodiment, the liquid pharmaceutical composition of the present invention does not contain any amino acid in addition to arginine and lysine.

In one embodiment, the liquid pharmaceutical composition of the present invention does not contain EDTA and proline. In one embodiment, the liquid pharmaceutical composition of the present invention does not contain DTPA and methionine.

In one embodiment, the liquid pharmaceutical composition comprises arginine and/or lysine in combination with a sugar. In one embodiment, the liquid pharmaceutical composition comprises arginine and/or lysine in combination with a non-reducing sugar. In one embodiment, the liquid pharmaceutical composition comprises arginine and/or lysine in combination with trehalose. In one embodiment, the liquid pharmaceutical composition comprises arginine and/or lysine in combination with sucrose.

The term “sugar” refers to an organic compound comprising only carbon, hydrogen, and oxygen, usually with a hydrogen:oxygen atom ratio of 2:1 and the empirical formula Cm(H2O)n. The term “sugar” includes mono-, di-, oligo- and polysaccharides. Examples of sugars include glucose, fructose, galactose, xylose, ribose, sucrose, mannose, lactose, maltose, trehalose, starch, and glycogen. Preferably, the sugar is a non-reducing sugar.

Non-reducing sugars are sugars which are not able to act as a reducing agent, as they do not comprise a free aldehyde or ketone group. Preferably, the non-reducing sugar is selected from sucrose and trehalose.

In one embodiment, the sugar is trehalose. Trehalose is a non-reducing sugar. It is a disaccharide formed by a 1,1-glycosidic bond between a glucose and a fructose unit. Preferably, the dihydrate form of trehalose is used. The concentration of trehalose dihydrate in the liquid pharmaceutical composition of the present invention is 80 mM to 200 mM, preferably the concentration of trehalose dihydrate is 90 mM to 180 mM, more preferably the concentration of trehalose dihydrate is 100 mM to 170 mM or 100 mM to 160 mM, even more preferably the concentration of trehalose dihydrate is 110 mM to 150 mM or 110 mM to 140 mM and most preferably the concentration of trehalose dihydrate is 120 mM.

In one embodiment, the sugar is sucrose. Sucrose is a non-reducing sugar. It is a disaccharide formed by a 1,2-glycosidic bond between two α-glucose units. The concentration of sucrose in the liquid pharmaceutical composition of the present invention is 80 mM to 200 mM, preferably the concentration of sucrose is 90 mM to 180 mM, more preferably the concentration of sucrose is 100 mM to 170 mM or 100 mM to 160 mM, even more preferably the concentration of sucrose is 110 mM to 150 mM or 110 mM to 140 mM and most preferably the concentration of sucrose is 120 mM.

In one embodiment, the liquid pharmaceutical composition of the present invention comprises arginine and trehalose dihydrate. In one embodiment, the liquid pharmaceutical composition of the present invention comprises 50 mM to 150 mM arginine and 80 mM to 200 mM trehalose dihydrate, preferably 55 mM to 140 mM or 55 mM to 130 mM or 55 mM to 120 mM arginine and 90 mM to 180 mM trehalose dihydrate, more preferably 60 mM to 110 mM or 60 mM to 100 mM or 60 mM to 90 mM arginine and 100 mM to 170 mM or 100 mM to 160 mM trehalose dihydrate, even more preferably 65 mM to 85 mM or 65 mM to 80 mM or 65 mM to 75 mM arginine and 110 mM to 150 mM or 110 mM to 140 mM trehalose dihydrate and most preferably the liquid pharmaceutical composition of the present invention comprises 70 mM arginine and 120 mM trehalose dihydrate.

In one embodiment, the liquid pharmaceutical composition of the present invention comprises lysine and trehalose dihydrate. In one embodiment, the liquid pharmaceutical composition of the present invention comprises 50 mM to 150 mM lysine and 80 mM to 200 mM trehalose dihydrate, preferably 55 mM to 140 mM or 55 mM to 130 mM or 55 mM to 120 mM lysine and 90 mM to 180 mM trehalose dihydrate, more preferably 60 mM to 110 mM or 60 mM to 100 mM or 60 mM to 90 mM lysine and 100 mM to 170 mM or 100 mM to 160 mM trehalose dihydrate, even more preferably 65 mM to 85 mM or 65 mM to 80 mM or 65 mM to 75 mM lysine and 110 mM to 150 mM or 110 mM to 140 mM trehalose dihydrate and most preferably the liquid pharmaceutical composition of the present invention comprises 70 mM lysine and 120 mM trehalose dihydrate.

In one embodiment, the liquid pharmaceutical composition of the present invention comprises arginine and sucrose. In one embodiment, the liquid pharmaceutical composition of the present invention comprises 50 mM to 150 mM arginine and 80 mM to 200 mM sucrose, preferably 55 mM to 140 mM or 55 mM to 130 mM or 55 mM to 120 mM arginine and 90 mM to 180 mM sucrose, more preferably 60 mM to 110 mM or 60 mM to 100 mM or 60 mM to 90 mM arginine and 100 mM to 170 mM or 100 mM to 160 mM sucrose, even more preferably 65 mM to 85 mM or 65 mM to 80 mM or 65 mM to 75 mM arginine and 110 mM to 150 mM or 110 mM to 140 mM sucrose and most preferably the liquid pharmaceutical composition of the present invention comprises 70 mM arginine and 120 mM sucrose.

In one embodiment, the liquid pharmaceutical composition of the present invention comprises lysine and sucrose. In one embodiment, the liquid pharmaceutical composition of the present invention comprises 50 mM to 150 mM lysine and 80 mM to 200 mM sucrose, preferably 55 mM to 140 mM or 55 mM to 130 mM or 55 mM to 120 mM lysine and 90 mM to 180 mM sucrose, more preferably 60 mM to 110 mM or 60 mM to 100 mM or 60 mM to 90 mM lysine and 100 mM to 170 mM or 100 mM to 160 mM sucrose, even more preferably 65 mM to 85 mM or 65 mM to 80 mM or 65 mM to 75 mM lysine and 110 mM to 150 mM or 110 mM to 140 mM sucrose and most preferably the liquid pharmaceutical composition of the present invention comprises 70 mM lysine and 120 mM sucrose.

The term “antibody” or “immunoglobulin” is used herein in the broadest sense and includes full length antibodies, genetically engineered antibodies, recombinant antibodies, multivalent antibodies, monoclonal antibodies, polyclonal antibodies, bispecific antibodies, multispecific antibodies, chimeric antibodies, humanized antibodies, fully human antibodies, as well as fragments of such antibodies as long as they remain functional and exhibit the desired biological activity. The “biological activity” of an antibody refers to the ability of the antibody to bind to antigen and result in a biological response which can be measured in vitro or in vivo.

A full length antibody comprises an antigen-binding variable region of the light (VL) and heavy chain (VH), a light chain constant region (CL) and heavy chain constant domains CH1, CH2 and CH3.

The term “antibody fragment” or “antigen-binding fragment” is used herein in the broadest sense and comprises a portion of a full length antibody, preferably comprising the antigen-binding or variable region thereof. An antibody fragment retains the original specificity of the parent immunoglobulin. Examples of antibody fragments include, e.g., Fab, Fab′, F(ab′)2, and Fv fragments, diabodies, linear antibodies, single-chain antibody molecules, and multispecific antibodies formed from antibody fragment(s).

A “monoclonal antibody” is an antibody that is specific for a single epitope of an antigen, i.e. directed against a single determinant on an antigen. Methods for producing monoclonal antibodies are known to the person skilled in the art.

The term “recombinant antibody” refers to all antibodies prepared, expressed, created or isolated by recombinant means, such as antibodies isolated from a transgenic host cell, such as e.g. a NS0 or CHO cell, or from an animal transgenic for immunoglobulin genes, or antibodies expressed using recombinant expression vectors transfected into a host cell, such as e.g. SP 2/0 mouse myeloma cells.

A “humanized antibody” is a human antibody wherein the antigen binding portion (CDR) is derived from non-human species, such as a mouse, and thus has a different specificity compared to the parent immunoglobulin. The CDR protein sequences can be modified to increase their similarities to antibody variants produced naturally in humans.

A “fully human antibody” is an antibody wherein all parts of the antibody including the antigen binding portion (CDR) are derived from human.

The term “anti-PD1 antibody” refers to an antibody that specifically binds to cell death protein 1 (PD-1) and inhibits the binding of PD-1 to its ligand PD-L1 and optionally inhibits the binding of PD-1 to its ligands PD-L1 and PD-L2. The anti-PD1 antibody thereby abolishes the suppressive effect of the PD-1/PD-L1 interaction on T cells. Known anti-PD1 antibodies include, but are not limited to, pembrolizumab, nivolumab, cemiplimab and cetrelimab.

Pembrolizumab (also known as MK-3475, SCH 900475 and lambrolizumab) is a humanized IgG4 mAb with the structure described in WHO Drug Information, Vol. 27, No. 2, pages 161-162 (2013). It comprises the light chain CDRs according to SEQ ID NOs: 1, 2 und 3 and the heavy chain CDRs according to SEQ ID NOs: 6, 7 and 8. The amino acid sequence of the light chain variable region is shown in SEQ ID NO: 4 and the amino acid sequence of the heavy chain variable region is shown in SEQ ID NO: 9. The amino acid sequence of the complete light chain is shown in SEQ ID NO: 5 and the amino acid sequence of the complete heavy chain is shown in SEQ ID NO: 10. Pembrolizumab has been approved inter alia for the treatment of patients with unresectable or metastatic melanoma and for the treatment of certain patients with recurrent or metastatic head and neck squamous cell cancer (HNSCC), classical Hodgkin lymphoma (cHL), urothelial carcinoma, gastric cancer, microsatellite instability-high (MSI-H) cancer and non-small cell lung cancer. The present commercial pembrolizumab formulation contains 10 mM histidine, 70 mg/ml sucrose, 0.2 mg/ml polysorbate 80 and water for injection, pH 5.5 and is supplied in a concentration of 25 mg/ml.

Nivolumab (also known as ONO-4538, BMS-936558, MDX1106) is a fully human monoclonal IgG4 antibody which comprises the heavy and light chain amino acid sequences and CDRs described in Table 2 of WO 2018/204368. Pembrolizumab has been approved for the treatment of patients with melanoma, renal carcinoma, non-small cell lung cancer and urothelial carcinoma. The present commercial nivolumab formulation contains 30 mg/ml mannitol, 0.008 mg/ml pentetic acid, 0.2 mg/ml polysorbate 80, 2.92 mg/ml sodium chloride, 5.88 mg/ml sodium citrate dihydrate, and water for injection, pH 6.0 and is supplied in a concentration of 10 mg/ml.

In one embodiment, the liquid pharmaceutical composition does not contain an anti-LAG3 or an anti-CTLA-4 antibody. In one embodiment, the anti-human PD-1 antibody is not a bispecific antibody binding to human PD-1 and another target.

In one embodiment, the anti-human PD-1 antibody is the only antibody present in the liquid pharmaceutical composition of the present invention. In one embodiment, pembrolizumab is the only antibody present in the liquid pharmaceutical composition of the present invention.

In one embodiment, the anti-human PD-1 antibody is the only pharmaceutically active agent present in the liquid pharmaceutical composition. In one embodiment, pembrolizumab is the only pharmaceutically active agent present in the liquid pharmaceutical composition.

In one embodiment, the concentration of the anti-PD1 antibody in the liquid pharmaceutical compositions of the present invention is 100 mg/ml to 200 mg/ml, preferably 110 mg/ml to 195 mg/ml or 120 mg/ml to 190 mg/ml or 130 mg/ml to 185 mg/ml, more preferably 140 mg/ml to 180 mg/ml or 145 mg/ml to 175 mg/ml or 150 mg/ml to 170 mg/ml or 155 mg/ml to 170 mg/ml or 160 mg/ml to 170 mg/ml and most preferably 165 mg/ml.

In one embodiment, the concentration of the anti-PD1 antibody in the liquid pharmaceutical compositions of the present invention is 10-80 mg/ml, preferably 15-70 mg/ml or 15-60 mg/ml, more preferably 20-50 mg/ml or 20-40 mg/ml, and most preferably 25 mg/ml. A liquid pharmaceutical composition having such an antibody concentration is suitable for intravenous administration.

In one embodiment, the liquid pharmaceutical composition of the present invention is isotonic, i.e. has essentially the same osmotic pressure as human blood which is typically in the range of 270 to 328 mOsm. Osmotic pressure can be determined, for example, using a vapor pressure or ice-freezing type osmometer.

In one embodiment, the liquid pharmaceutical composition of the present invention has a low viscosity which is required for subcutaneous administration. In one embodiment, the liquid pharmaceutical composition of the present invention has a shear viscosity of less than 20 mPa*sec, preferably of less than 19 mPa*sec, more preferably less than 18 mPa*sec and most preferably of less than 17 mPa*sec. In one embodiment, the liquid pharmaceutical composition of the present invention has a shear viscosity of 10 to 20 mPa*sec, preferably of 11 to 19 mPa*sec, more preferably of 12 to 18 mPa*sec and most preferably of 13 to 17 mPa*sec.

In one embodiment, the liquid pharmaceutical composition of the present invention has a low viscosity which is required for subcutaneous administration. In one embodiment, the concentration of the anti-PD1 antibody in the liquid pharmaceutical compositions of the present invention is 165 mg/ml and the liquid pharmaceutical composition of the present invention has a shear viscosity of less than 20 mPa*sec, preferably of less than 19 mPa*sec, more preferably less than 18 mPa*sec and most preferably of less than 17 mPa*sec. In one embodiment, the concentration of the anti-PD1 antibody in the liquid pharmaceutical compositions of the present invention is 165 mg/ml and the liquid pharmaceutical composition of the present invention has a shear viscosity of 10 to 20 mPa*sec, preferably of 11 to 19 mPa*sec, more preferably of 12 to 18 mPa*sec and most preferably of 13 to 17 mPa*sec.

In one embodiment, the liquid pharmaceutical composition of the present invention has a low viscosity which is required for subcutaneous administration. In one embodiment, the concentration of pembrolizumab in the liquid pharmaceutical compositions of the present invention is 165 mg/ml and the liquid pharmaceutical composition of the present invention has a shear viscosity of less than 20 mPa*sec, preferably of less than 19 mPa*sec, more preferably less than 18 mPa*sec and most preferably of less than 17 mPa*sec. In one embodiment, the concentration of pembrolizumab in the liquid pharmaceutical compositions of the present invention is 165 mg/ml and the liquid pharmaceutical composition of the present invention has a shear viscosity of 10 to 20 mPa*sec, preferably of 11 to 19 mPa*sec, more preferably of 12 to 18 mPa*sec and most preferably of 13 to 17 mPa*sec. The pharmaceutical compositions of the present invention can be used in the treatment of cancer, in particular in the treatment of melanoma, non-small cell lung carcinoma, classical Hodgkin lymphoma, urothelial carcinoma, head and neck squamous cell carcinoma, renal cell carcinoma, colorectal cancer, oesophageal carcinoma, small cell lung cancer, microsatellite instability-high or mismatch repair deficient cancer, primary mediastinal large B-cell lymphoma, gastric or gastroesophageal junction adenocarcinoma, hepatocellular carcinoma, Merkel cell carcinoma, endometrial carcinoma, tumor mutational burden-high cancer, cutaneous squamous cell carcinoma, triple-negative breast cancer or cervical cancer.

The pharmaceutical compositions of the present invention may contain further active agents, in particular further anti-tumor agents such as chemotherapeutics. Examples of such chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, es tram us tine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammall and calicheamicin phill); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomopholino-doxorubicin, 2-pyrrolino-doxorubicin and deoxydoxorubicin), epirubicin, esorubicin, idarubicin, marcellomycin, mitomy cins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY1 17018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

In an alternative embodiment, the pharmaceutical compositions of the present invention may be administered in combination with any of the chemotherapeutics listed above, but the chemotherapeutic is present in a separate pharmaceutical composition.

In an alternative embodiment, the pharmaceutical composition of the present invention is to be administered with pemetrexed and platinum-based chemotherapy. In an alternative embodiment, the pharmaceutical composition of the present invention is to be administered with pemetrexed and platinum-based chemotherapy in the treatment of non-small cell lung carcinoma. In an alternative embodiment, the pharmaceutical composition of the present invention is to be administered with carboplatin and either paclitaxel or nab-paclitaxel. In an alternative embodiment, the pharmaceutical composition of the present invention is to be administered with carboplatin and either paclitaxel or nab-paclitaxel in the treatment of non-small cell lung carcinoma.

In an alternative embodiment, the pharmaceutical composition of the present invention is to be administered with platinum-based chemotherapy and 5-fluorouracil. In an alternative embodiment, the pharmaceutical composition of the present invention is to be administered with platinum-based chemotherapy and 5-fluorouracil in the treatment of head and neck squamous cell carcinoma.

In an alternative embodiment, the pharmaceutical composition of the present invention is to be administered with axitinib. In an alternative embodiment, the pharmaceutical composition of the present invention is to be administered with axitinib in the treatment of renal cell carcinoma.

In an alternative embodiment, the pharmaceutical composition of the present invention is to be administered with platinum- and fluoropyrimidine-based chemotherapy. In an alternative embodiment, the pharmaceutical composition of the present invention is to be administered with platinum- and fluoropyrimidine-based chemotherapy in the treatment of oesophageal carcinoma.

In an alternative embodiment, the pharmaceutical composition of the present invention is to be administered with lenvatinib. In an alternative embodiment, the pharmaceutical composition of the present invention is to be administered with lenvatinib in the treatment of endometrial carcinoma or renal cell carcinoma.

In an alternative embodiment, the pharmaceutical composition of the present invention is to be administered with chemotherapy. In an alternative embodiment, the pharmaceutical composition of the present invention is to be administered with chemotherapy in the treatment of triple-negative breast cancer.

In an alternative embodiment, the pharmaceutical composition of the present invention is to be administered with chemotherapy with or without bevacizumab. In an alternative embodiment, the pharmaceutical composition of the present invention is to be administered with chemotherapy with or without bevacizumab in the treatment of cervical cancer.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml mg/ml of an anti-human PD1 antibody; 100 mM to 200 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml mg/ml of an anti-human PD1 antibody; 100 mM to 200 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml mg/ml of an anti-human PD1 antibody; 100 mM to 200 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 100 to 200 mg/ml mg/ml of an anti-human PD1 antibody; 100 mM to 200 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 1 to 50 mM of succinate buffer, pH 5.5. In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 1 to 50 mM of succinate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 1 to 50 mM of succinate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 1 to 50 mM of succinate buffer, pH 5.5.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of a non-ionic surfactant and 1 to 50 mM of succinate buffer, pH 5.5.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of a non-ionic surfactant and 1 to 50 mM of succinate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 100 to 200 mg/ml mg/ml of pembrolizumab; 100 mM to 200 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 100 to 200 mg/ml mg/ml of pembrolizumab; 120 mM to 180 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of a non-ionic surfactant and 1 to 50 mM of succinate buffer, pH 5.5.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of a non-ionic surfactant and 1 to 50 mM of succinate buffer, pH 5.5.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of a non-ionic surfactant and 1 to 50 mM of succinate buffer, pH 5.5.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 165 mg/ml of an anti-human PD1 antibody; 50 mM to 350 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 165 mg/ml of an anti-human PD1 antibody; 100 mM to 200 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 165 mg/ml of an anti-human PD1 antibody; 120 mM to 180 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 165 mg/ml of pembrolizumab; 50 mM to 350 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 165 mg/ml of pembrolizumab; 100 mM to 200 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 165 mg/ml of pembrolizumab; 120 mM to 180 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 165 mg/ml of pembrolizumab; 50 mM to 350 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 165 mg/ml of pembrolizumab; 100 mM to 200 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 165 mg/ml of pembrolizumab; 120 mM to 180 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 165 mg/ml of pembrolizumab; 50 mM to 350 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 165 mg/ml of pembrolizumab; 100 mM to 200 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 165 mg/ml of pembrolizumab; 120 mM to 180 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 165 mg/ml of pembrolizumab; 50 mM to 350 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 165 mg/ml of pembrolizumab; 100 mM to 200 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 165 mg/ml of pembrolizumab; 120 mM to 180 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 165 mg/ml of pembrolizumab; 50 mM to 350 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 165 mg/ml of pembrolizumab; 100 mM to 200 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 165 mg/ml of pembrolizumab; 120 mM to 180 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 165 mg/ml of pembrolizumab; 50 mM to 350 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 165 mg/ml of pembrolizumab; 100 mM to 200 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 165 mg/ml of pembrolizumab; 120 mM to 180 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 165 mg/ml of pembrolizumab; 50 mM to 350 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 165 mg/ml of pembrolizumab; 100 mM to 200 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 165 mg/ml of pembrolizumab; 120 mM to 180 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 165 mg/ml of pembrolizumab; 50 mM to 350 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 165 mg/ml of pembrolizumab; 100 mM to 200 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 165 mg/ml of pembrolizumab; 120 mM to 180 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 165 mg/ml of pembrolizumab; 50 mM to 350 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 165 mg/ml of pembrolizumab; 100 mM to 200 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 165 mg/ml of pembrolizumab; 120 mM to 180 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 165 mg/ml of pembrolizumab; 50 mM to 350 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 165 mg/ml of pembrolizumab; 100 mM to 200 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 165 mg/ml of pembrolizumab; 120 mM to 180 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 165 mg/ml of pembrolizumab; 50 mM to 350 mM of arginine; 0.1 mg/ml to 0.4 mg/ml of a non-ionic surfactant and 1 to 50 mM of succinate buffer, pH 5.5.

In one embodiment, the present invention relates to a liquid pharmaceutical composition comprising: 165 mg/ml of pembrolizumab; 100 mM to 200 mM of arginine; 0.1 mg/ml to 0.4 mg/ml of a non-ionic surfactant and 1 to 50 mM of succinate buffer, pH 5.5.

In one embodiment, the present invention relates to a liquid pharmaceutical composition comprising: 165 mg/ml of pembrolizumab; 120 mM to 180 mM of arginine; 0.1 mg/ml to 0.4 mg/ml of a non-ionic surfactant and 1 to 50 mM of succinate buffer, pH 5.5.

In one embodiment, the present invention relates to a liquid pharmaceutical composition comprising: 165 mg/ml of pembrolizumab; 50 mM to 350 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 165 mg/ml of pembrolizumab; 100 mM to 200 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 165 mg/ml of pembrolizumab; 120 mM to 180 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 165 mg/ml of pembrolizumab; 50 mM to 350 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 165 mg/ml of pembrolizumab; 100 mM to 200 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 165 mg/ml of pembrolizumab; 120 mM to 180 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 165 mg/ml of pembrolizumab; 50 mM to 350 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 165 mg/ml of pembrolizumab; 100 mM to 200 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 165 mg/ml of pembrolizumab; 120 mM to 180 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 165 mg/ml of pembrolizumab; 50 mM to 350 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 165 mg/ml of pembrolizumab; 100 mM to 200 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 165 mg/ml of pembrolizumab; 120 mM to 180 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 165 mg/ml of pembrolizumab; 50 mM to 350 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 165 mg/ml of pembrolizumab; 100 mM to 200 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 165 mg/ml of pembrolizumab; 120 mM to 180 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 165 mg/ml of pembrolizumab; 50 mM to 350 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 165 mg/ml of pembrolizumab; 100 mM to 200 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 165 mg/ml of pembrolizumab; 120 mM to 180 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 165 mg/ml of pembrolizumab; 50 mM to 350 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 165 mg/ml of pembrolizumab; 100 mM to 200 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 165 mg/ml of pembrolizumab; 120 mM to 180 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 165 mg/ml of pembrolizumab; 50 mM to 350 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 165 mg/ml of pembrolizumab; 100 mM to 200 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 165 mg/ml of pembrolizumab; 120 mM to 180 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 165 mg/ml of pembrolizumab; 50 mM to 350 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 165 mg/ml of pembrolizumab; 100 mM to 200 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 165 mg/ml of pembrolizumab; 120 mM to 180 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 165 mg/ml of pembrolizumab; 50 mM to 350 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 165 mg/ml of pembrolizumab; 100 mM to 200 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 165 mg/ml of pembrolizumab; 120 mM to 180 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 165 mg/ml of pembrolizumab; 50 mM to 350 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 165 mg/ml of pembrolizumab; 100 mM to 200 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 165 mg/ml of pembrolizumab; 120 mM to 180 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 165 mg/ml of pembrolizumab; 50 mM to 350 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 165 mg/ml of pembrolizumab; 100 mM to 200 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 165 mg/ml of pembrolizumab; 120 mM to 180 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 165 mg/ml of pembrolizumab; 50 mM to 350 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 165 mg/ml of pembrolizumab; 100 mM to 200 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 165 mg/ml of pembrolizumab; 120 mM to 180 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of a non-ionic surfactant and 1 to 50 mM of succinate buffer, pH 5.5.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 100 to 200 mg/ml of an anti-human PD1 antibody; 150 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 100 to 200 mg/ml of pembrolizumab; 150 mM of arginine; 0.1 mg/ml to 0.4 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml of pembrolizumab; 150 mM of arginine; 0.1 mg/ml to 0.4 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml of pembrolizumab; 150 mM of arginine; 0.1 mg/ml to 0.4 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml of pembrolizumab; 150 mM of arginine; 0.1 mg/ml to 0.4 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml of pembrolizumab; 150 mM of arginine; 0.1 mg/ml to 0.4 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml of pembrolizumab; 150 mM of arginine; 0.1 mg/ml to 0.4 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml of pembrolizumab; 150 mM of arginine; 0.1 mg/ml to 0.4 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml of pembrolizumab; 150 mM of arginine; 0.1 mg/ml to 0.4 mg/ml of polysorbate 80 and 1 to 50 mM of malate buffer, pH 5.0 to 5.8.

In one embodiment, the present invention relates to a liquid pharmaceutical composition comprising: 100 to 200 mg/ml of pembrolizumab; 150 mM of arginine; 0.1 mg/ml to 0.4 mg/ml of a non-ionic surfactant and 1 to 50 mM of acetate buffer, pH 5.5.

In one embodiment, the present invention relates to a liquid pharmaceutical composition comprising: 100 to 200 mg/ml of pembrolizumab; 150 mM of arginine; 0.1 mg/ml to 0.4 mg/ml of a non-ionic surfactant and 1 to 50 mM of adipate buffer, pH 5.5.

In one embodiment, the present invention relates to a liquid pharmaceutical composition comprising: 100 to 200 mg/ml of pembrolizumab; 150 mM of arginine; 0.1 mg/ml to 0.4 mg/ml of a non-ionic surfactant and 1 to 50 mM of succinate buffer, pH 5.5.

In one embodiment, the present invention relates to a liquid pharmaceutical composition comprising: 100 to 200 mg/ml of pembrolizumab; 150 mM of arginine; 0.1 mg/ml to 0.4 mg/ml of a non-ionic surfactant and 1 to 50 mM of malate buffer, pH 5.5.

In one embodiment, the present invention relates to a liquid pharmaceutical composition comprising: 100 to 200 mg/ml of pembrolizumab; 150 mM of arginine; 0.1 mg/ml to 0.4 mg/ml of a non-ionic surfactant and 1 to 50 mM of acetate buffer, pH 5.2.

In one embodiment, the present invention relates to a liquid pharmaceutical composition comprising: 100 to 200 mg/ml of pembrolizumab; 150 mM of arginine; 0.1 mg/ml to 0.4 mg/ml of a non-ionic surfactant and 1 to 50 mM of adipate buffer, pH 5.2.

In one embodiment, the present invention relates to a liquid pharmaceutical composition comprising: 100 to 200 mg/ml of pembrolizumab; 150 mM of arginine; 0.1 mg/ml to 0.4 mg/ml of a non-ionic surfactant and 1 to 50 mM of succinate buffer, pH 5.2.

In one embodiment, the present invention relates to a liquid pharmaceutical composition comprising: 100 to 200 mg/ml of pembrolizumab; 150 mM of arginine; 0.1 mg/ml to 0.4 mg/ml of a non-ionic surfactant and 1 to 50 mM of malate buffer, pH 5.2.

In one embodiment, the present invention relates to a liquid pharmaceutical composition comprising: 100 to 200 mg/ml of pembrolizumab; 150 mM of arginine; 0.1 mg/ml to 0.4 mg/ml of polysorbate 80 and 1 to 50 mM of acetate buffer, pH 5.5.

In one embodiment, the present invention relates to a liquid pharmaceutical composition comprising: 100 to 200 mg/ml of pembrolizumab; 150 mM of arginine; 0.1 mg/ml to 0.4 mg/ml of polysorbate 80 and 1 to 50 mM of adipate buffer, pH 5.5.

In one embodiment, the present invention relates to a liquid pharmaceutical composition comprising: 100 to 200 mg/ml of pembrolizumab; 150 mM of arginine; 0.1 mg/ml to 0.4 mg/ml of polysorbate 80 and 1 to 50 mM of succinate buffer, pH 5.5.

In one embodiment, the present invention relates to a liquid pharmaceutical composition comprising: 100 to 200 mg/ml of pembrolizumab; 150 mM of arginine; 0.1 mg/ml to 0.4 mg/ml of polysorbate 80 and 1 to 50 mM of malate buffer, pH 5.5.

In one embodiment, the present invention relates to a liquid pharmaceutical composition comprising: 100 to 200 mg/ml of pembrolizumab; 150 mM of arginine; 0.1 mg/ml to 0.4 mg/ml of polysorbate 80 and 1 to 50 mM of acetate buffer, pH 5.2.

In one embodiment, the present invention relates to a liquid pharmaceutical composition comprising: 100 to 200 mg/ml of pembrolizumab; 150 mM of arginine; 0.1 mg/ml to 0.4 mg/ml of polysorbate 80 and 1 to 50 mM of adipate buffer, pH 5.2.

In one embodiment, the present invention relates to a liquid pharmaceutical composition comprising: 100 to 200 mg/ml of pembrolizumab; 150 mM of arginine; 0.1 mg/ml to 0.4 mg/ml of polysorbate 80 and 1 to 50 mM of succinate buffer, pH 5.2.

In one embodiment, the present invention relates to a liquid pharmaceutical composition comprising: 100 to 200 mg/ml of pembrolizumab; 150 mM of arginine; 0.1 mg/ml to 0.4 mg/ml of polysorbate 80 and 1 to 50 mM of malate buffer, pH 5.2.

In one embodiment, the present invention relates to a liquid pharmaceutical composition comprising: 100 to 200 mg/ml of an anti-human PD1 antibody; 50 mM to 350 mM of arginine; 0.2 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.2 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.2 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.2 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.2 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.2 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.2 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.2 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.2 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.2 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.2 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.2 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.2 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.2 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.2 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.2 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.2 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.2 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.2 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.2 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.2 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.2 mg/ml of polysorbate 80 and 1 to 50 mM of malate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.2 mg/ml of polysorbate 80 and 1 to 50 mM of malate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.2 mg/ml of polysorbate 80 and 1 to 50 mM of malate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.2 mg/ml of a non-ionic surfactant and 1 to 50 mM of acetate buffer, pH 5.5.

In one embodiment, the present invention relates to a liquid pharmaceutical composition comprising: 100 to 200 mg/ml of an anti-human PD1 antibody; 100 mM to 200 mM of arginine; 0.2 mg/ml of a non-ionic surfactant and 1 to 50 mM of acetate buffer, pH 5.5.

In one embodiment, the present invention relates to a liquid pharmaceutical composition comprising: 100 to 200 mg/ml of an anti-human PD1 antibody; 120 mM to 180 mM of arginine; 0.2 mg/ml of a non-ionic surfactant and 1 to 50 mM of acetate buffer, pH 5.5.

In one embodiment, the present invention relates to a liquid pharmaceutical composition comprising: 100 to 200 mg/ml of an anti-human PD1 antibody; 50 mM to 350 mM of arginine; 0.2 mg/ml of a non-ionic surfactant and 1 to 50 mM of adipate buffer, pH 5.5.

In one embodiment, the present invention relates to a liquid pharmaceutical composition comprising: 100 to 200 mg/ml of an anti-human PD1 antibody; 100 mM to 200 mM of arginine; 0.2 mg/ml of a non-ionic surfactant and 1 to 50 mM of adipate buffer, pH 5.5.

In one embodiment, the present invention relates to a liquid pharmaceutical composition comprising: 100 to 200 mg/ml of an anti-human PD1 antibody; 120 mM to 180 mM of arginine; 0.2 mg/ml of a non-ionic surfactant and 1 to 50 mM of adipate buffer, pH 5.5.

In one embodiment, the present invention relates to a liquid pharmaceutical composition comprising: 100 to 200 mg/ml of an anti-human PD1 antibody; 50 mM to 350 mM of arginine; 0.2 mg/ml of a non-ionic surfactant and 1 to 50 mM of succinate buffer, pH 5.5.

In one embodiment, the present invention relates to a liquid pharmaceutical composition comprising: 100 to 200 mg/ml of an anti-human PD1 antibody; 100 mM to 200 mM of arginine; 0.2 mg/ml of a non-ionic surfactant and 1 to 50 mM of succinate buffer, pH 5.5.

In one embodiment, the present invention relates to a liquid pharmaceutical composition comprising: 100 to 200 mg/ml of an anti-human PD1 antibody; 120 mM to 180 mM of arginine; 0.2 mg/ml of a non-ionic surfactant and 1 to 50 mM of succinate buffer, pH 5.5.

In one embodiment, the present invention relates to a liquid pharmaceutical composition comprising: 100 to 200 mg/ml of an anti-human PD1 antibody; 50 mM to 350 mM of arginine; 0.2 mg/ml of a non-ionic surfactant and 1 to 50 mM of malate buffer, pH 5.5.

In one embodiment, the present invention relates to a liquid pharmaceutical composition comprising: 100 to 200 mg/ml of an anti-human PD1 antibody; 100 mM to 200 mM of arginine; 0.2 mg/ml of a non-ionic surfactant and 1 to 50 mM of malate buffer, pH 5.5.

In one embodiment, the present invention relates to a liquid pharmaceutical composition comprising: 100 to 200 mg/ml of an anti-human PD1 antibody; 120 mM to 180 mM of arginine; 0.2 mg/ml of a non-ionic surfactant and 1 to 50 mM of malate buffer, pH 5.5.

In one embodiment, the present invention relates to a liquid pharmaceutical composition comprising: 100 to 200 mg/ml of an anti-human PD1 antibody; 50 mM to 350 mM of arginine; 0.2 mg/ml of a non-ionic surfactant and 1 to 50 mM of acetate buffer, pH 5.2.

In one embodiment, the present invention relates to a liquid pharmaceutical composition comprising: 100 to 200 mg/ml of an anti-human PD1 antibody; 100 mM to 200 mM of arginine; 0.2 mg/ml of a non-ionic surfactant and 1 to 50 mM of acetate buffer, pH 5.2.

In one embodiment, the present invention relates to a liquid pharmaceutical composition comprising: 100 to 200 mg/ml of an anti-human PD1 antibody; 120 mM to 180 mM of arginine; 0.2 mg/ml of a non-ionic surfactant and 1 to 50 mM of acetate buffer, pH 5.2.

In one embodiment, the present invention relates to a liquid pharmaceutical composition comprising: 100 to 200 mg/ml of an anti-human PD1 antibody; 50 mM to 350 mM of arginine; 0.2 mg/ml of a non-ionic surfactant and 1 to 50 mM of adipate buffer, pH 5.2.

In one embodiment, the present invention relates to a liquid pharmaceutical composition comprising: 100 to 200 mg/ml of an anti-human PD1 antibody; 100 mM to 200 mM of arginine; 0.2 mg/ml of a non-ionic surfactant and 1 to 50 mM of adipate buffer, pH 5.2.

In one embodiment, the present invention relates to a liquid pharmaceutical composition comprising: 100 to 200 mg/ml of an anti-human PD1 antibody; 120 mM to 180 mM of arginine; 0.2 mg/ml of a non-ionic surfactant and 1 to 50 mM of adipate buffer, pH 5.2.

In one embodiment, the present invention relates to a liquid pharmaceutical composition comprising: 100 to 200 mg/ml of an anti-human PD1 antibody; 50 mM to 350 mM of arginine; 0.2 mg/ml of a non-ionic surfactant and 1 to 50 mM of succinate buffer, pH 5.2.

In one embodiment, the present invention relates to a liquid pharmaceutical composition comprising: 100 to 200 mg/ml of an anti-human PD1 antibody; 100 mM to 200 mM of arginine; 0.2 mg/ml of a non-ionic surfactant and 1 to 50 mM of succinate buffer, pH 5.2.

In one embodiment, the present invention relates to a liquid pharmaceutical composition comprising: 100 to 200 mg/ml of an anti-human PD1 antibody; 120 mM to 180 mM of arginine; 0.2 mg/ml of a non-ionic surfactant and 1 to 50 mM of succinate buffer, pH 5.2.

In one embodiment, the present invention relates to a liquid pharmaceutical composition comprising: 100 to 200 mg/ml of an anti-human PD1 antibody; 50 mM to 350 mM of arginine; 0.2 mg/ml of a non-ionic surfactant and 1 to 50 mM of malate buffer, pH 5.2.

In one embodiment, the present invention relates to a liquid pharmaceutical composition comprising: 100 to 200 mg/ml of an anti-human PD1 antibody; 100 mM to 200 mM of arginine; 0.2 mg/ml of a non-ionic surfactant and 1 to 50 mM of malate buffer, pH 5.2.

In one embodiment, the present invention relates to a liquid pharmaceutical composition comprising: 100 to 200 mg/ml of an anti-human PD1 antibody; 120 mM to 180 mM of arginine; 0.2 mg/ml of a non-ionic surfactant and 1 to 50 mM of malate buffer, pH 5.2.

In one embodiment, the present invention relates to a liquid pharmaceutical composition comprising: 100 to 200 mg/ml of an anti-human PD1 antibody; 50 mM to 350 mM of arginine; 0.2 mg/ml of polysorbate 80 and 1 to 50 mM of acetate buffer, pH 5.5.

In one embodiment, the present invention relates to a liquid pharmaceutical composition comprising: 100 to 200 mg/ml of an anti-human PD1 antibody; 100 mM to 200 mM of arginine; 0.2 mg/ml of polysorbate 80 and 1 to 50 mM of acetate buffer, pH 5.5.

In one embodiment, the present invention relates to a liquid pharmaceutical composition comprising: 100 to 200 mg/ml of an anti-human PD1 antibody; 120 mM to 180 mM of arginine; 0.2 mg/ml of polysorbate 80 and 1 to 50 mM of acetate buffer, pH 5.5.

In one embodiment, the present invention relates to a liquid pharmaceutical composition comprising: 100 to 200 mg/ml of an anti-human PD1 antibody; 50 mM to 350 mM of arginine; 0.2 mg/ml of polysorbate 80 and 1 to 50 mM of adipate buffer, pH 5.5.

In one embodiment, the present invention relates to a liquid pharmaceutical composition comprising: 100 to 200 mg/ml of an anti-human PD1 antibody; 100 mM to 200 mM of arginine; 0.2 mg/ml of polysorbate 80 and 1 to 50 mM of adipate buffer, pH 5.5.

In one embodiment, the present invention relates to a liquid pharmaceutical composition comprising: 100 to 200 mg/ml of an anti-human PD1 antibody; 120 mM to 180 mM of arginine; 0.2 mg/ml of polysorbate 80 and 1 to 50 mM of adipate buffer, pH 5.5.

In one embodiment, the present invention relates to a liquid pharmaceutical composition comprising: 100 to 200 mg/ml of an anti-human PD1 antibody; 50 mM to 350 mM of arginine; 0.2 mg/ml of polysorbate 80 and 1 to 50 mM of succinate buffer, pH 5.5.

In one embodiment, the present invention relates to a liquid pharmaceutical composition comprising: 100 to 200 mg/ml of an anti-human PD1 antibody; 100 mM to 200 mM of arginine; 0.2 mg/ml of polysorbate 80 and 1 to 50 mM of succinate buffer, pH 5.5.

In one embodiment, the present invention relates to a liquid pharmaceutical composition comprising: 100 to 200 mg/ml of an anti-human PD1 antibody; 120 mM to 180 mM of arginine; 0.2 mg/ml of polysorbate 80 and 1 to 50 mM of succinate buffer, pH 5.5.

In one embodiment, the present invention relates to a liquid pharmaceutical composition comprising: 100 to 200 mg/ml of an anti-human PD1 antibody; 50 mM to 350 mM of arginine; 0.2 mg/ml of polysorbate 80 and 1 to 50 mM of malate buffer, pH 5.5.

In one embodiment, the present invention relates to a liquid pharmaceutical composition comprising: 100 to 200 mg/ml of an anti-human PD1 antibody; 100 mM to 200 mM of arginine; 0.2 mg/ml of polysorbate 80 and 1 to 50 mM of malate buffer, pH 5.5.

In one embodiment, the present invention relates to a liquid pharmaceutical composition comprising: 100 to 200 mg/ml of an anti-human PD1 antibody; 120 mM to 180 mM of arginine; 0.2 mg/ml of polysorbate 80 and 1 to 50 mM of malate buffer, pH 5.5.

In one embodiment, the present invention relates to a liquid pharmaceutical composition comprising: 100 to 200 mg/ml of an anti-human PD1 antibody; 50 mM to 350 mM of arginine; 0.2 mg/ml of polysorbate 80 and 1 to 50 mM of acetate buffer, pH 5.2.

In one embodiment, the present invention relates to a liquid pharmaceutical composition comprising: 100 to 200 mg/ml of an anti-human PD1 antibody; 100 mM to 200 mM of arginine; 0.2 mg/ml of polysorbate 80 and 1 to 50 mM of acetate buffer, pH 5.2.

In one embodiment, the present invention relates to a liquid pharmaceutical composition comprising: 100 to 200 mg/ml of an anti-human PD1 antibody; 120 mM to 180 mM of arginine; 0.2 mg/ml of polysorbate 80 and 1 to 50 mM of acetate buffer, pH 5.2.

In one embodiment, the present invention relates to a liquid pharmaceutical composition comprising: 100 to 200 mg/ml of an anti-human PD1 antibody; 50 mM to 350 mM of arginine; 0.2 mg/ml of polysorbate 80 and 1 to 50 mM of adipate buffer, pH 5.2.

In one embodiment, the present invention relates to a liquid pharmaceutical composition comprising: 100 to 200 mg/ml of an anti-human PD1 antibody; 100 mM to 200 mM of arginine; 0.2 mg/ml of polysorbate 80 and 1 to 50 mM of adipate buffer, pH 5.2.

In one embodiment, the present invention relates to a liquid pharmaceutical composition comprising: 100 to 200 mg/ml of an anti-human PD1 antibody; 120 mM to 180 mM of arginine; 0.2 mg/ml of polysorbate 80 and 1 to 50 mM of adipate buffer, pH 5.2.

In one embodiment, the present invention relates to a liquid pharmaceutical composition comprising: 100 to 200 mg/ml of an anti-human PD1 antibody; 50 mM to 350 mM of arginine; 0.2 mg/ml of polysorbate 80 and 1 to 50 mM of succinate buffer, pH 5.2.

In one embodiment, the present invention relates to a liquid pharmaceutical composition comprising: 100 to 200 mg/ml of an anti-human PD1 antibody; 100 mM to 200 mM of arginine; 0.2 mg/ml of polysorbate 80 and 1 to 50 mM of succinate buffer, pH 5.2.

In one embodiment, the present invention relates to a liquid pharmaceutical composition comprising: 100 to 200 mg/ml of an anti-human PD1 antibody; 120 mM to 180 mM of arginine; 0.2 mg/ml of polysorbate 80 and 1 to 50 mM of succinate buffer, pH 5.2.

In one embodiment, the present invention relates to a liquid pharmaceutical composition comprising: 100 to 200 mg/ml of an anti-human PD1 antibody; 50 mM to 350 mM of arginine; 0.2 mg/ml of polysorbate 80 and 1 to 50 mM of malate buffer, pH 5.2.

In one embodiment, the present invention relates to a liquid pharmaceutical composition comprising: 100 to 200 mg/ml of an anti-human PD1 antibody; 100 mM to 200 mM of arginine; 0.2 mg/ml of polysorbate 80 and 1 to 50 mM of malate buffer, pH 5.2.

In one embodiment, the present invention relates to a liquid pharmaceutical composition comprising: 100 to 200 mg/ml of an anti-human PD1 antibody; 120 mM to 180 mM of arginine; 0.2 mg/ml of polysorbate 80 and 1 to 50 mM of malate buffer, pH 5.2.

In one embodiment, the present invention relates to a liquid pharmaceutical composition comprising: 100 to 200 mg/ml of pembrolizumab; 50 mM to 350 mM of arginine; 0.2 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.2 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.2 mg/ml of a non-ionic surfactant and 1 to 50 mM of acetate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.2 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.2 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.2 mg/ml of a non-ionic surfactant and 1 to 50 mM of adipate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.2 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.2 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.2 mg/ml of a non-ionic surfactant and 1 to 50 mM of succinate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.2 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.2 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.2 mg/ml of a non-ionic surfactant and 1 to 50 mM of malate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.2 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.2 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.2 mg/ml of polysorbate 80 and 1 to 50 mM of acetate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.2 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.2 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.2 mg/ml of polysorbate 80 and 1 to 50 mM of adipate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.2 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.2 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.2 mg/ml of polysorbate 80 and 1 to 50 mM of succinate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.2 mg/ml of polysorbate 80 and 1 to 50 mM of malate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.2 mg/ml of polysorbate 80 and 1 to 50 mM of malate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.2 mg/ml of polysorbate 80 and 1 to 50 mM of malate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.2 mg/ml of a non-ionic surfactant and 1 to 50 mM of acetate buffer, pH 5.5.

In one embodiment, the present invention relates to a liquid pharmaceutical composition comprising: 100 to 200 mg/ml of pembrolizumab; 100 mM to 200 mM of arginine; 0.2 mg/ml of a non-ionic surfactant and 1 to 50 mM of acetate buffer, pH 5.5.

In one embodiment, the present invention relates to a liquid pharmaceutical composition comprising: 100 to 200 mg/ml of pembrolizumab; 120 mM to 180 mM mM of arginine; 0.2 mg/ml of a non-ionic surfactant and 1 to 50 mM of acetate buffer, pH 5.5.

In one embodiment, the present invention relates to a liquid pharmaceutical composition comprising: 100 to 200 mg/ml of pembrolizumab; 50 mM to 350 mM of arginine; 0.2 mg/ml of a non-ionic surfactant and 1 to 50 mM of adipate buffer, pH 5.5.

In one embodiment, the present invention relates to a liquid pharmaceutical composition comprising: 100 to 200 mg/ml of pembrolizumab; 100 mM to 200 mM of arginine; 0.2 mg/ml of a non-ionic surfactant and 1 to 50 mM of adipate buffer, pH 5.5.

In one embodiment, the present invention relates to a liquid pharmaceutical composition comprising: 100 to 200 mg/ml of pembrolizumab; 120 mM to 180 mM mM of arginine; 0.2 mg/ml of a non-ionic surfactant and 1 to 50 mM of adipate buffer, pH 5.5.

In one embodiment, the present invention relates to a liquid pharmaceutical composition comprising: 100 to 200 mg/ml of pembrolizumab; 50 mM to 350 mM of arginine; 0.2 mg/ml of a non-ionic surfactant and 1 to 50 mM of succinate buffer, pH 5.5.

In one embodiment, the present invention relates to a liquid pharmaceutical composition comprising: 100 to 200 mg/ml of pembrolizumab; 100 mM to 200 mM of arginine; 0.2 mg/ml of a non-ionic surfactant and 1 to 50 mM of succinate buffer, pH 5.5.

In one embodiment, the present invention relates to a liquid pharmaceutical composition comprising: 100 to 200 mg/ml of pembrolizumab; 120 mM to 180 mM mM of arginine; 0.2 mg/ml of a non-ionic surfactant and 1 to 50 mM of succinate buffer, pH 5.5.

In one embodiment, the present invention relates to a liquid pharmaceutical composition comprising: 100 to 200 mg/ml of pembrolizumab; 50 mM to 350 mM of arginine; 0.2 mg/ml of a non-ionic surfactant and 1 to 50 mM of malate buffer, pH 5.5.

In one embodiment, the present invention relates to a liquid pharmaceutical composition comprising: 100 to 200 mg/ml of pembrolizumab; 100 mM to 200 mM of arginine; 0.2 mg/ml of a non-ionic surfactant and 1 to 50 mM of malate buffer, pH 5.5.

In one embodiment, the present invention relates to a liquid pharmaceutical composition comprising: 100 to 200 mg/ml of pembrolizumab; 120 mM to 180 mM mM of arginine; 0.2 mg/ml of a non-ionic surfactant and 1 to 50 mM of malate buffer, pH 5.5.

In one embodiment, the present invention relates to a liquid pharmaceutical composition comprising: 100 to 200 mg/ml of pembrolizumab; 50 mM to 350 mM of arginine; 0.2 mg/ml of a non-ionic surfactant and 1 to 50 mM of acetate buffer, pH 5.2.

In one embodiment, the present invention relates to a liquid pharmaceutical composition comprising: 100 to 200 mg/ml of pembrolizumab; 100 mM to 200 mM of arginine; 0.2 mg/ml of a non-ionic surfactant and 1 to 50 mM of acetate buffer, pH 5.2.

In one embodiment, the present invention relates to a liquid pharmaceutical composition comprising: 100 to 200 mg/ml of pembrolizumab; 120 mM to 180 mM of arginine; 0.2 mg/ml of a non-ionic surfactant and 1 to 50 mM of acetate buffer, pH 5.2.

In one embodiment, the present invention relates to a liquid pharmaceutical composition comprising: 100 to 200 mg/ml of pembrolizumab; 50 mM to 350 mM of arginine; 0.2 mg/ml of a non-ionic surfactant and 1 to 50 mM of adipate buffer, pH 5.2.

In one embodiment, the present invention relates to a liquid pharmaceutical composition comprising: 100 to 200 mg/ml of pembrolizumab; 100 mM to 200 mM of arginine; 0.2 mg/ml of a non-ionic surfactant and 1 to 50 mM of adipate buffer, pH 5.2.

In one embodiment, the present invention relates to a liquid pharmaceutical composition comprising: 100 to 200 mg/ml of pembrolizumab; 120 mM to 180 mM mM of arginine; 0.2 mg/ml of a non-ionic surfactant and 1 to 50 mM of adipate buffer, pH 5.2.

In one embodiment, the present invention relates to a liquid pharmaceutical composition comprising: 100 to 200 mg/ml of pembrolizumab; 50 mM to 350 mM of arginine; 0.2 mg/ml of a non-ionic surfactant and 1 to 50 mM of succinate buffer, pH 5.2.

In one embodiment, the present invention relates to a liquid pharmaceutical composition comprising: 100 to 200 mg/ml of pembrolizumab; 100 mM to 200 mM of arginine; 0.2 mg/ml of a non-ionic surfactant and 1 to 50 mM of succinate buffer, pH 5.2.

In one embodiment, the present invention relates to a liquid pharmaceutical composition comprising: 100 to 200 mg/ml of pembrolizumab; 120 mM to 180 mM of arginine; 0.2 mg/ml of a non-ionic surfactant and 1 to 50 mM of succinate buffer, pH 5.2.

In one embodiment, the present invention relates to a liquid pharmaceutical composition comprising: 100 to 200 mg/ml of pembrolizumab; 50 mM to 350 mM of arginine; 0.2 mg/ml of a non-ionic surfactant and 1 to 50 mM of malate buffer, pH 5.2.

In one embodiment, the present invention relates to a liquid pharmaceutical composition comprising: 100 to 200 mg/ml of pembrolizumab; 100 mM to 200 mM of arginine; 0.2 mg/ml of a non-ionic surfactant and 1 to 50 mM of malate buffer, pH 5.2.

In one embodiment, the present invention relates to a liquid pharmaceutical composition comprising: 100 to 200 mg/ml of pembrolizumab; 120 mM to 180 mM of arginine; 0.2 mg/ml of a non-ionic surfactant and 1 to 50 mM of malate buffer, pH 5.2.

In one embodiment, the present invention relates to a liquid pharmaceutical composition comprising: 100 to 200 mg/ml of pembrolizumab; 50 mM to 350 mM of arginine; 0.2 mg/ml of polysorbate 80 and 1 to 50 mM of acetate buffer, pH 5.5.

In one embodiment, the present invention relates to a liquid pharmaceutical composition comprising: 100 to 200 mg/ml of pembrolizumab; 100 mM to 200 mM of arginine; 0.2 mg/ml of polysorbate 80 and 1 to 50 mM of acetate buffer, pH 5.5.

In one embodiment, the present invention relates to a liquid pharmaceutical composition comprising: 100 to 200 mg/ml of pembrolizumab; 120 mM to 180 mM of arginine; 0.2 mg/ml of polysorbate 80 and 1 to 50 mM of acetate buffer, pH 5.5.

In one embodiment, the present invention relates to a liquid pharmaceutical composition comprising: 100 to 200 mg/ml of pembrolizumab; 50 mM to 350 mM of arginine; 0.2 mg/ml of polysorbate 80 and 1 to 50 mM of adipate buffer, pH 5.5.

In one embodiment, the present invention relates to a liquid pharmaceutical composition comprising: 100 to 200 mg/ml of pembrolizumab; 100 mM to 200 mM of arginine; 0.2 mg/ml of polysorbate 80 and 1 to 50 mM of adipate buffer, pH 5.5.

In one embodiment, the present invention relates to a liquid pharmaceutical composition comprising: 100 to 200 mg/ml of pembrolizumab; 120 mM to 180 mM of arginine; 0.2 mg/ml of polysorbate 80 and 1 to 50 mM of adipate buffer, pH 5.5.

In one embodiment, the present invention relates to a liquid pharmaceutical composition comprising: 100 to 200 mg/ml of pembrolizumab; 50 mM to 350 mM of arginine; 0.2 mg/ml of polysorbate 80 and 1 to 50 mM of succinate buffer, pH 5.5.

In one embodiment, the present invention relates to a liquid pharmaceutical composition comprising: 100 to 200 mg/ml of pembrolizumab; 100 mM to 200 mM of arginine; 0.2 mg/ml of polysorbate 80 and 1 to 50 mM of succinate buffer, pH 5.5.

In one embodiment, the present invention relates to a liquid pharmaceutical composition comprising: 100 to 200 mg/ml of pembrolizumab; 120 mM to 180 mM of arginine; 0.2 mg/ml of polysorbate 80 and 1 to 50 mM of succinate buffer, pH 5.5.

In one embodiment, the present invention relates to a liquid pharmaceutical composition comprising: 100 to 200 mg/ml of pembrolizumab; 50 mM to 350 mM of arginine; 0.2 mg/ml of polysorbate 80 and 1 to 50 mM of malate buffer, pH 5.5.

In one embodiment, the present invention relates to a liquid pharmaceutical composition comprising: 100 to 200 mg/ml of pembrolizumab; 100 mM to 200 mM of arginine; 0.2 mg/ml of polysorbate 80 and 1 to 50 mM of malate buffer, pH 5.5.

In one embodiment, the present invention relates to a liquid pharmaceutical composition comprising: 100 to 200 mg/ml of pembrolizumab; 120 mM to 180 mM of arginine; 0.2 mg/ml of polysorbate 80 and 1 to 50 mM of malate buffer, pH 5.5.

In one embodiment, the present invention relates to a liquid pharmaceutical composition comprising: 100 to 200 mg/ml of pembrolizumab; 50 mM to 350 mM of arginine; 0.2 mg/ml of polysorbate 80 and 1 to 50 mM of acetate buffer, pH 5.2.

In one embodiment, the present invention relates to a liquid pharmaceutical composition comprising: 100 to 200 mg/ml of pembrolizumab; 100 mM to 200 mM of arginine; 0.2 mg/ml of polysorbate 80 and 1 to 50 mM of acetate buffer, pH 5.2.

In one embodiment, the present invention relates to a liquid pharmaceutical composition comprising: 100 to 200 mg/ml of pembrolizumab; 120 mM to 180 mM of arginine; 0.2 mg/ml of polysorbate 80 and 1 to 50 mM of acetate buffer, pH 5.2.

In one embodiment, the present invention relates to a liquid pharmaceutical composition comprising: 100 to 200 mg/ml of pembrolizumab; 50 mM to 350 mM of arginine; 0.2 mg/ml of polysorbate 80 and 1 to 50 mM of adipate buffer, pH 5.2.

In one embodiment, the present invention relates to a liquid pharmaceutical composition comprising: 100 to 200 mg/ml of pembrolizumab; 100 mM to 200 mM of arginine; 0.2 mg/ml of polysorbate 80 and 1 to 50 mM of adipate buffer, pH 5.2.

In one embodiment, the present invention relates to a liquid pharmaceutical composition comprising: 100 to 200 mg/ml of pembrolizumab; 120 mM to 180 mM of arginine; 0.2 mg/ml of polysorbate 80 and 1 to 50 mM of adipate buffer, pH 5.2.

In one embodiment, the present invention relates to a liquid pharmaceutical composition comprising: 100 to 200 mg/ml of pembrolizumab; 50 mM to 350 mM of arginine; 0.2 mg/ml of polysorbate 80 and 1 to 50 mM of succinate buffer, pH 5.2.

In one embodiment, the present invention relates to a liquid pharmaceutical composition comprising: 100 to 200 mg/ml of pembrolizumab; 100 mM to 200 mM of arginine; 0.2 mg/ml of polysorbate 80 and 1 to 50 mM of succinate buffer, pH 5.2.

In one embodiment, the present invention relates to a liquid pharmaceutical composition comprising: 100 to 200 mg/ml of pembrolizumab; 120 mM to 180 mM of arginine; 0.2 mg/ml of polysorbate 80 and 1 to 50 mM of succinate buffer, pH 5.2.

In one embodiment, the present invention relates to a liquid pharmaceutical composition comprising: 100 to 200 mg/ml of pembrolizumab; 50 mM to 350 mM of arginine; 0.2 mg/ml of polysorbate 80 and 1 to 50 mM of malate buffer, pH 5.2.

In one embodiment, the present invention relates to a liquid pharmaceutical composition comprising: 100 to 200 mg/ml of pembrolizumab; 100 mM to 200 mM of arginine; 0.2 mg/ml of polysorbate 80 and 1 to 50 mM of malate buffer, pH 5.2.

In one embodiment, the present invention relates to a liquid pharmaceutical composition comprising: 100 to 200 mg/ml of pembrolizumab; 120 mM to 180 mM of arginine; 0.2 mg/ml of polysorbate 80 and 1 to 50 mM of malate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 10 mM of acetate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 10 mM of acetate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 10 mM of acetate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 10 mM of adipate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 10 mM of adipate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 10 mM of adipate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 10 mM of succinate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 10 mM of succinate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 10 mM of succinate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 10 mM of malate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 10 mM of malate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 10 mM of malate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 10 mM of acetate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 10 mM of acetate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 10 mM of acetate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 10 mM of adipate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 10 mM of adipate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 10 mM of adipate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 10 mM of succinate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 10 mM of succinate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 10 mM of succinate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 10 mM of malate buffer, pH 5.0 to 5.8.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 10 mM of malate buffer, pH 5.0 to 5.8.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 10 mM of malate buffer, pH 5.0 to 5.8.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 10 mM of acetate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 10 mM of acetate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 10 mM of acetate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 10 mM of adipate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 10 mM of adipate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 10 mM of adipate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 10 mM of succinate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 10 mM of succinate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 10 mM of succinate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 10 mM of malate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 10 mM of malate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 10 mM of malate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 10 mM of acetate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 10 mM of acetate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 10 mM of acetate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 10 mM of adipate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 10 mM of adipate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 10 mM of adipate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 10 mM of succinate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 10 mM of succinate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 10 mM of succinate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of a non-ionic surfactant and 10 mM of malate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of a non-ionic surfactant and 10 mM of malate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of a non-ionic surfactant and 10 mM of malate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 10 mM of acetate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 10 mM of acetate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 10 mM of acetate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 10 mM of adipate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 10 mM of adipate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 10 mM of adipate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 10 mM of succinate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 10 mM of succinate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 10 mM of succinate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 10 mM of malate buffer, pH 5.5.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 10 mM of malate buffer, pH 5.5.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 10 mM of malate buffer, pH 5.5.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 10 mM of acetate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 10 mM of acetate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 10 mM of acetate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 10 mM of adipate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 10 mM of adipate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 10 mM of adipate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 10 mM of succinate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 10 mM of succinate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 10 mM of succinate buffer, pH 5.2.

In one embodiment, the present invention relates to a liquid pharmaceutical composition comprising: 100 to 200 mg/ml of an anti-human PD1 antibody; 50 mM to 350 mM of arginine; 0.1 mg/ml to 0.4 mg/ml of polysorbate 80 and 10 mM of malate buffer, pH 5.2.

In one embodiment, the present invention relates to a liquid pharmaceutical composition comprising: 100 to 200 mg/ml of an anti-human PD1 antibody; 100 mM to 200 mM of arginine; 0.1 mg/ml to 0.4 mg/ml of polysorbate 80 and 10 mM of malate buffer, pH 5.2.

In one embodiment, the present invention relates to a liquid pharmaceutical composition comprising: 100 to 200 mg/ml of an anti-human PD1 antibody; 120 mM to 180 mM of arginine; 0.1 mg/ml to 0.4 mg/ml of polysorbate 80 and 10 mM of malate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 10 mM of acetate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of a non-ionic surfactant and 10 mM of acetate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of a non-ionic surfactant and 10 mM of acetate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 10 mM of adipate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of a non-ionic surfactant and 10 mM of adipate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of a non-ionic surfactant and 10 mM of adipate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 10 mM of succinate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of a non-ionic surfactant and 10 mM of succinate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of a non-ionic surfactant and 10 mM of succinate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 10 mM of malate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of a non-ionic surfactant and 10 mM of malate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of a non-ionic surfactant and 10 mM of malate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 10 mM of acetate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of polysorbate 80 and 10 mM of acetate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of polysorbate 80 and 10 mM of acetate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 10 mM of adipate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of polysorbate 80 and 10 mM of adipate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of polysorbate 80 and 10 mM of adipate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 10 mM of succinate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of polysorbate 80 and 10 mM of succinate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of polysorbate 80 and 10 mM of succinate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 10 mM of malate buffer, pH 5.0 to 5.8.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of polysorbate 80 and 10 mM of malate buffer, pH 5.0 to 5.8.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of polysorbate 80 and 10 mM of malate buffer, pH 5.0 to 5.8.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 10 mM of acetate buffer, pH 5.5.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of a non-ionic surfactant and 10 mM of acetate buffer, pH 5.5.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of a non-ionic surfactant and 10 mM of acetate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 10 mM of adipate buffer, pH 5.5.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of a non-ionic surfactant and 10 mM of adipate buffer, pH 5.5.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of a non-ionic surfactant and 10 mM of adipate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 10 mM of succinate buffer, pH 5.5.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of a non-ionic surfactant and 10 mM of succinate buffer, pH 5.5.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of a non-ionic surfactant and 10 mM of succinate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 10 mM of malate buffer, pH 5.5.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of a non-ionic surfactant and 10 mM of malate buffer, pH 5.5.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of a non-ionic surfactant and 10 mM of malate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 10 mM of acetate buffer, pH 5.2.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of a non-ionic surfactant and 10 mM of acetate buffer, pH 5.2.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of a non-ionic surfactant and 10 mM of acetate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 10 mM of adipate buffer, pH 5.2.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of a non-ionic surfactant and 10 mM of adipate buffer, pH 5.2.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of a non-ionic surfactant and 10 mM of adipate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 10 mM of succinate buffer, pH 5.2.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of a non-ionic surfactant and 10 mM of succinate buffer, pH 5.2.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of a non-ionic surfactant and 10 mM of succinate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of a non-ionic surfactant and 10 mM of malate buffer, pH 5.2.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of a non-ionic surfactant and 10 mM of malate buffer, pH 5.2.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of a non-ionic surfactant and 10 mM of malate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 10 mM of acetate buffer, pH 5.5.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of polysorbate 80 and 10 mM of acetate buffer, pH 5.5.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of polysorbate 80 and 10 mM of acetate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 10 mM of adipate buffer, pH 5.5.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of polysorbate 80 and 10 mM of adipate buffer, pH 5.5.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of polysorbate 80 and 10 mM of adipate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 10 mM of succinate buffer, pH 5.5.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of polysorbate 80 and 10 mM of succinate buffer, pH 5.5.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of polysorbate 80 and 10 mM of succinate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 10 mM of malate buffer, pH 5.5.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of polysorbate 80 and 10 mM of malate buffer, pH 5.5.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of polysorbate 80 and 10 mM of malate buffer, pH 5.5.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 10 mM of acetate buffer, pH 5.2.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of polysorbate 80 and 10 mM of acetate buffer, pH 5.2.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of polysorbate 80 and 10 mM of acetate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 10 mM of adipate buffer, pH 5.2.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of polysorbate 80 and 10 mM of adipate buffer, pH 5.2.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of polysorbate 80 and 10 mM of adipate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 10 mM of succinate buffer, pH 5.2.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of polysorbate 80 and 10 mM of succinate buffer, pH 5.2.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of polysorbate 80 and 10 mM of succinate buffer, pH 5.2.

In one embodiment, the present invention relates to a liquid pharmaceutical composition comprising: 100 to 200 mg/ml of pembrolizumab; 50 mM to 350 mM of arginine; 0.1 mg/ml to 0.4 mg/ml of polysorbate 80 and 10 mM of malate buffer, pH 5.2.

In one embodiment, the present invention relates to a liquid pharmaceutical composition comprising: 100 to 200 mg/ml of pembrolizumab; 100 mM to 200 mM of arginine; 0.1 mg/ml to 0.4 mg/ml of polysorbate 80 and 10 mM of malate buffer, pH 5.2.

In one embodiment, the present invention relates to a liquid pharmaceutical composition comprising: 100 to 200 mg/ml of pembrolizumab; 120 mM to 180 mM of arginine; 0.1 mg/ml to 0.4 mg/ml of polysorbate 80 and 10 mM of malate buffer, pH 5.2.

In one embodiment, the present invention relates to a liquid pharmaceutical composition comprising: 165 mg/ml of an anti-PD1 antibody; 150 mM arginine; 0.2 mg/ml polysorbate 80 and 10 mM of acetate buffer, pH 5.0 to 5.8.

In one embodiment, the present invention relates to a liquid pharmaceutical composition comprising: 165 mg/ml of an anti-PD1 antibody; 150 mM arginine; 0.2 mg/ml polysorbate 80 and 10 mM of adipate buffer, pH 5.5.

In one embodiment, the present invention relates to a liquid pharmaceutical composition comprising: 165 mg/ml of an anti-PD1 antibody; 150 mM arginine; 0.2 mg/ml polysorbate 80 and 10 mM of succinate buffer, pH 5.5.

In one embodiment, the present invention relates to a liquid pharmaceutical composition comprising: 165 mg/ml of an anti-PD1 antibody; 150 mM arginine; 0.2 mg/ml polysorbate 80 and 10 mM of malate buffer, pH 5.5.

In one embodiment, the present invention relates to a liquid pharmaceutical composition comprising: 165 mg/ml of an anti-PD1 antibody; 150 mM arginine; 0.2 mg/ml polysorbate 80 and 10 mM of acetate buffer, pH 5.2.

In one embodiment, the present invention relates to a liquid pharmaceutical composition comprising: 165 mg/ml of an anti-PD1 antibody; 150 mM arginine; 0.2 mg/ml polysorbate 80 and 10 mM of adipate buffer, pH 5.2.

In one embodiment, the present invention relates to a liquid pharmaceutical composition comprising: 165 mg/ml of an anti-PD1 antibody; 150 mM arginine; 0.2 mg/ml polysorbate 80 and 10 mM of succinate buffer, pH 5.2.

In one embodiment, the present invention relates to a liquid pharmaceutical composition comprising: 165 mg/ml of an anti-PD1 antibody; 150 mM arginine; 0.2 mg/ml polysorbate 80 and 10 mM of malate buffer, pH 5.2.

In one embodiment, the present invention relates to a liquid pharmaceutical composition comprising: 165 mg/ml of pembrolizumab; 150 mM arginine; 0.2 mg/ml polysorbate 80 and 10 mM of acetate buffer, pH 5.0 to 5.8.

In one embodiment, the present invention relates to a liquid pharmaceutical composition comprising: 165 mg/ml of pembrolizumab; 150 mM arginine; 0.2 mg/ml polysorbate 80 and 10 mM of adipate buffer, pH 5.0 to 5.8.

In one embodiment, the present invention relates to a liquid pharmaceutical composition comprising: 165 mg/ml of pembrolizumab; 150 mM arginine; 0.2 mg/ml polysorbate 80 and 10 mM of succinate buffer, pH 5.0 to 5.8.

In one embodiment, the present invention relates to a liquid pharmaceutical composition comprising: 165 mg/ml of pembrolizumab; 150 mM arginine; 0.2 mg/ml polysorbate 80 and 10 mM of malate buffer, pH 5.0 to 5.8.

In one embodiment, the present invention relates to a liquid pharmaceutical composition comprising: 165 mg/ml of pembrolizumab; 150 mM arginine; 0.2 mg/ml polysorbate 80 and 10 mM of acetate buffer, pH 5.5.

In one embodiment, the present invention relates to a liquid pharmaceutical composition comprising: 165 mg/ml of pembrolizumab; 150 mM arginine; 0.2 mg/ml polysorbate 80 and 10 mM of adipate buffer, pH 5.5.

In one embodiment, the present invention relates to a liquid pharmaceutical composition comprising: 165 mg/ml of pembrolizumab; 150 mM arginine; 0.2 mg/ml polysorbate 80 and 10 mM of succinate buffer, pH 5.5.

In one embodiment, the present invention relates to a liquid pharmaceutical composition comprising: 165 mg/ml of pembrolizumab; 150 mM arginine; 0.2 mg/ml polysorbate 80 and 10 mM of malate buffer, pH 5.5.

In one embodiment, the present invention relates to a liquid pharmaceutical composition comprising: 165 mg/ml of pembrolizumab; 150 mM arginine; 0.2 mg/ml polysorbate 80 and 10 mM of acetate buffer, pH 5.2.

In one embodiment, the present invention relates to a liquid pharmaceutical composition comprising: 165 mg/ml of pembrolizumab; 150 mM arginine; 0.2 mg/ml polysorbate 80 and 10 mM of adipate buffer, pH 5.2.

In one embodiment, the present invention relates to a liquid pharmaceutical composition comprising: 165 mg/ml of pembrolizumab; 150 mM arginine; 0.2 mg/ml polysorbate 80 and 10 mM of succinate buffer, pH 5.2.

In one embodiment, the present invention relates to a liquid pharmaceutical composition comprising: 165 mg/ml of pembrolizumab; 150 mM arginine; 0.2 mg/ml polysorbate 80 and 10 mM of malate buffer, pH 5.2.

In one embodiment, the present invention relates to a liquid pharmaceutical composition consisting of acetate buffer, L-arginine, polysorbate 80, pembrolizumab and water for injection and having a pH of 5.5.

In one embodiment, the present invention relates to a liquid pharmaceutical composition consisting of acetate buffer, L-arginine, polysorbate 80, pembrolizumab and water for injection and having a pH of 5.2.

In one embodiment, the present invention relates to a liquid pharmaceutical composition consisting of adipate buffer, L-arginine, polysorbate 80, pembrolizumab and water for injection and having a pH of 5.5.

In one embodiment, the present invention relates to a liquid pharmaceutical composition consisting of adipate buffer, L-lysine, trehalose dihydrate, polysorbate 80, pembrolizumab and water for injection and having a pH of 5.5.

In one embodiment, the present invention relates to a liquid pharmaceutical composition consisting of succinate buffer, L-arginine, polysorbate 80, pembrolizumab and water for injection and having a pH of 5.5.

In one embodiment, the present invention relates to a liquid pharmaceutical composition consisting of 10 mM acetate buffer, 150 mM L-arginine, 0.2 mg/ml polysorbate 80, 165 mg/ml pembrolizumab and water for injection and having a pH of 5.5.

In one embodiment, the present invention relates to a liquid pharmaceutical composition consisting of 10 mM acetate buffer, 150 mM L-arginine, 0.2 mg/ml polysorbate 80, 165 mg/ml pembrolizumab and water for injection and having a pH of 5.2.

In one embodiment, the present invention relates to a liquid pharmaceutical composition consisting of 10 mM adipate buffer, 150 mM L-arginine, 0.2 mg/ml polysorbate 80, 165 mg/ml pembrolizumab and water for injection and having a pH of 5.5.

In one embodiment, the present invention relates to a liquid pharmaceutical composition consisting of 10 mM adipate buffer, 70 mM L-lysine, 120 mM trehalose dihydrate, 0.2 mg/ml polysorbate 80, 165 mg/ml pembrolizumab and water for injection and having a pH of 5.5.

In one embodiment, the present invention relates to a liquid pharmaceutical composition consisting of 10 mM succinate buffer, 150 mM L-arginine, 0.2 mg/ml polysorbate 80, 165 mg/ml pembrolizumab and water for injection and having a pH of 5.5.

In one embodiment, the present invention relates to a liquid pharmaceutical composition consisting of 10 mM acetate buffer, 140 mM L-lysine, 0.2 mg/ml polysorbate 80, 165 mg/ml pembrolizumab and water for injection and having a pH of 5.5.

In one embodiment, the present invention relates to a liquid pharmaceutical composition consisting of acetate buffer, L-lysine, polysorbate 80, pembrolizumab and water for injection and having a pH of 5.2 or 5.5.

In one embodiment, the present invention relates to a liquid pharmaceutical composition consisting of adipate buffer, L-lysine, polysorbate 80, pembrolizumab and water for injection and having a pH of 5.5.

In one embodiment, the present invention relates to a liquid pharmaceutical composition consisting of succinate buffer, L-lysine, polysorbate 80, pembrolizumab and water for injection and having a pH of 5.5.

In one embodiment, the present invention relates to a liquid pharmaceutical composition consisting of 10 mM acetate buffer, 140 mM L-lysine, 0.2 mg/ml polysorbate 80, 165 mg/ml pembrolizumab and water for injection and having a pH of 5.2.

In one embodiment, the present invention relates to a liquid pharmaceutical composition consisting of 10 mM adipate buffer, 140 mM L-lysine, 0.2 mg/ml polysorbate 80, 165 mg/ml pembrolizumab and water for injection and having a pH of 5.5.

In one embodiment, the present invention relates to a liquid pharmaceutical composition consisting of 10 mM succinate buffer, 140 mM L-lysine, 0.2 mg/ml polysorbate 80, 165 mg/ml pembrolizumab and water for injection and having a pH of 5.5.

The pharmaceutical compositions may be supplied in a vial or in a pre-filled syringe. The pharmaceutical compositions may be administered by intravenous infusion, e.g. over a period of 30 minutes or less.

Alternatively, the pharmaceutical compositions may be administered by subcutaneous injection. In this case, the concentration of the anti-PD1 antibody and preferably of pembrolizumab is 100 mg/ml to 200 mg/ml, preferably 110 mg/ml to 190 mg/ml or 120 mg/ml to 180 mg/ml, more preferably 130 mg/ml to 175 mg/ml or 140 mg/ml to 170 mg/ml and most preferably 165 mg/ml. In one embodiment, the concentration of the anti-PD1 antibody and preferably of pembrolizumab is 165 mg/ml.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

The detailed description is merely exemplary in nature and is not intended to limit application and uses. The following examples further illustrate the present invention without, however, limiting the scope of the invention thereto. Various changes and modifications can be made by those skilled in the art on the basis of the description of the invention, and such changes and modifications are also included in the present invention.

### EXAMPLES

### Formulation Development of 165 mg/mL Pembrolizumab Formulations

### Example 1: Method Panel

The following method panel for analytical characterization of highly concentrated pembrolizumab formulations was used completely or partially in example 2, example 3 and example 4:

a. Analysis of Protein Content by UV-VIS

The protein concentration was determined by absorption spectroscopy at 280 nm with correction for any aggregated particles at 320 nm using a Nanophotometer N120 from Implen.

Formulated drug substance was diluted with the respective placebo solution and measured against the respective placebo solution. Protein concentrations were calculated using the absorption coefficient of 1.418 L*g⁻¹*cm⁻¹. Concentrations were reported based on the mean of three replicate measurements.

b. Analysis of Chemical Modifications by Ilon Exchange Chromatography (IEX-HPLC)

For all formulated samples 30 μg of pembrolizumab were separated on a Thermo MabPAc SCX-10 BioLC Analytical CEX-column (074625, 4 mm×250 mm, 10 μm) using a Thermo Ultimate 3000 HPLC system. The mobile phase A consisted of 20 mmol/L MES, pH 6.2. The mobile phase B was composed of 20 mmol/L MES, 120 mmol/L KCl, pH 6.2. Elution was performed by applying a gradient as shown in Table 1.

TABLE 1
Gradient for IEX-HPLC analysis

| Time [min] | % A | & B | step |
|---|---|---|---|
| 0 | 90 | 10 | Injection |
| 2.5 | 80 | 20 | Quick pre-gradient |
| 12.5 | 55 | 45 | Main separation step |
| 12.5 | 0 | 100 | Flush Elution |
| 14 | 0 | 100 | |
| 14 | 90 | 10 | Re-equilibration |
| 17 | 90 | 10 | |

Data were recorded at 280 nm by a UV detector. Chromatogram profiles were compared, and main peak, acidic variants and basic variants were quantified. All measurements were performed in duplicates.
c. Analysis of High Molecular Weight Species (HMWS) by Size Exclusion Chromatography (SE-HPLC)

5 μg of formulated pembrolizumab (diluted to 1 mg/mL) were separated on a TSKgel® UP-SW3000 SEC column (4.6×150 mm, 2 μm, from Tosoh Bioscience) using a ThermoFisher Ultimate 3000 HPLC system with an isocratic flow of 0.35 mL/min (100 mmol/L sodium phosphate, 100 mmol/L sodium sulfate, 0.05% sodium azide, pH 6.7) to detect the presence of high molecular weight species of pembrolizumab (HMWS). Data were recorded at 280 nm by a UV detector. Chromatogram profiles were compared and evaluated for HMWS and main peak relative area. All measurements were performed in duplicates.

d. Analysis of Low Molecular Weight Species (LMWS) by Non-Reducing cGE (LabChip)

Non-reducing capillary gel electrophoresis (cGE) was performed to quantify LMWS in pembrolizumab formulations, using a Perkin Elmer Labchip GXII Touch Protein Characterization System, a HT Protein Express Chip (760499, Perkin Elmer) and Protein Express assay reagent kit (CLS960008, Perkin Elmer). The samples were prepared according to the manufacturer's general instruction as described in the Kit handbook (PN CLS140156, Rev. D) and in the following: Formulated pembrolizumab samples were diluted with water to a concentration of 2 mg/mL and 10 μl thereof were further diluted with 35 μl of denaturation buffer. The denaturation solution was composed of sample buffer (from reagent kit), LDS (L4632, from Sigma Aldrich) and NEM (E1271, from Sigma Aldrich). Sample denaturation was performed at 75° C. for 10 min in a Bio-Rad real-time PCR cycler. The samples were analyzed in triplicate wells at the assay concentration of 0.091 μg/μl. Electrophoretic separation was performed using the LabChip GXII Touch HT software, running the preset P200 Antibody Analysis (Perkin Elmer). Electropherogram profiles as well as purity (main peak) and impurity (LMWS, HMWS) content were monitored.

e. Determination of Turbity by Light Extinction at 350 nm

Turbidity of samples was analyzed by extinction at 350 nm using a Tecan Spark plate reader. Turbidity was measured in 384-well plates (781801, Greiner) by comparing the measured extinction to standards with standards referring to Ph. Eur 10.8, chapter 2.2.1.

f. Determination Subvisible Particles

Particle sizes in the subvisible range were determined by flow imaging analysis using a FlowCam 8100 from Yokogawa Fluid Imaging Technologies. For each measurement 120 μl of the solutions were introduced into the system with 100 μl imaged by the system. The data were grouped by particle size according to European Pharmacopoeia/US Pharmacopoeia into subvisible particles below 5 μm, between 5 μm and 10 μm, between 10.1 μm and 25 μm, and above 25 μm.

g. Visual Appearance

The visual appearance of samples was assessed using an inspection light box equipped with non-flickering fluorescent lamps and a black and a white background plate (Portable Inspection Hood MIH-PORT, Bosch). The samples were evaluated in their storage container without magnification to assess sample appearance with respect to visible particles, color and clarity.

h. PH Measurement

The pH was determined with 150 μl aliquots of the samples and either a calibrated Ultra Micro ISM pH-electrode or a Micro Pro-ISM pH-electrode (both Mettler Toledo) connected to a SevenExcellence pH-Meter (Mettler Toledo) at room temperature. Samples were equilibrated at room temperature for at least 30 min before measurement.

i. Determination of Dynamic Viscosity

Measurements were performed at 20° C. on a Kinexus ultra plus rheometer at 20° C. and a shear rate of 250 s-1. 60 μl, using a sample volume of 60 μl and a CP1/20 geometry. All samples were analyzed as single measurements.

### Example 2: Screening of Pembrolizumab Formulations—Stabilizers and Viscosity Reducing Agents

a. Sample Preparation

A short-term stability study at 40° C. for up to 2 weeks including mechanical stress and freeze/thaw stress was performed in order to screen for suitable formulations for highly concentrated pembrolizumab.

Several excipients were systematically screened to evaluate their effects on both physico-chemical stabilization as well as viscosity reduction of the solution, a well-known problem for generating highly concentrated solutions of drugs, but required to allow the subcutaneous route of application.

The following excipients were tested in formulations shown in Table 2: trehalose dihydrate, L-arginine, L-lysine, L-proline, L-phenylalanine, L-glycine and sodium chloride. These excipients were formulated in solutions comprising a target pH of 5.5, containing also 5 mmol/L citric acid as buffering excipient, 0.2 mg/mL polysorbate 80 as surfactant and a concentration range of pembrolizumab of 145 mg/mL to 170 mg/mL. In addition, arginine was titrated with either HCL or phosphoric acid in order to evaluate effects of the counterions chloride versus phosphate in combination with arginine. Concentrations of stabilizers were adjusted in a way to provide a solution osmolality of 300 mOsm/kg±50 mOsm/kg, allowing for subcutaneous injection with limited sensation of pain.

Pembrolizumab with a concentration of about 28 mg/mL, formulated in 10 mmol/L citrate, 300 mmol/L arginine at pH 5.5 (surfactant free) was used as starting material and 17 new formulations as listed in Table 2 were created with a Big Tuna automated buffer exchange system (Unchained Labs, Pleasanton, CA), using a 30 kDa MWCO filter made of polypropylene with regenerated cellulose and providing at least 96% formulation exchange.

After that pembrolizumab concentrations were increased and finally the solutions were spiked with polysorbate 80 to the target concentration.

The formulations shown in Table 2 were initially analyzed for protein concentration by UV-VIS spectroscopy at 280 nm and correct pH adjustment before starting an accelerated aging stability program. Storage conditions are shown in Table 3. In brief, samples were placed on storage for up to 2 weeks at 40° C./75% relative humidity (RH). In addition, samples were subjected to two conditions of mechanical stressing (overhead rotation, orbital shaking) as well as five freeze/thaw cycles (−80° C./+25° C.).

TABLE 2
Detailed information of formulations prepared within this study

| | | | Stabilizer 1/ tonicity | Stabilizer 2/ tonicity | | |
|---|---|---|---|---|---|---|
| No. | Pembrolizumab | Buffer system¹⁾ | agent 1 | agent 2 | Surfactant | pH |
| (1) | 153.2 mg/mL | 5 mmol/L citrate | 205 mmol/L trehalose | n.a. | 0.2 mg/mL polysorbate 80 | 5.5 |
| | | | dihydrate | | | |
| (2) | 170.9 mg/mL | 5 mmol/L citrate | 120 mmol/L trehalose | 60 mmol/L L-arginine | 0.2 mg/mL polysorbate 80 | 5.6 |
| | | | dihydrate | | | |
| (3) | 155.9 mg/mL | 5 mmol/L citrate | 80 mmol/L trehalose | 80 mmol/L L-arginine | 0.2 mg/mL polysorbate 80 | 5.5 |
| | | | dihydrate | | | |
| (4) | 153.4 mg/mL | 5 mmol/L citrate | n.a. | 120 mmol/L L-arginine | 0.2 mg/mL polysorbate 80 | 5.6 |
| (5) | 173.1 mg/mL | 5 mmol/L citrate | n.a. | 60 mmol/L L-arginine, | 0.2 mg/mL polysorbate 80 | 5.6 |
| | | | | 60 mmol/L L-lysine | | |
| (6) | 156.2 mg/mL | 5 mmol/L citrate | 120 mmol/L trehalose | 60 mmol/L L-lysine | 0.2 mg/mL polysorbate 80 | 5.5 |
| | | | dihydrate | | | |
| (7) | 154.2 mg/mL | 5 mmol/L citrate | 80 mmol/L trehalose | 80 mmol/L L-arginine | 0.2 mg/mL polysorbate 80 | 5.5 |
| | | | dihydrate | | | |
| (8) | 158.6 mg/mL | 5 mmol/L citrate | 170 mmol/L trehalose | 34 mmol/L L-arginine | 0.2 mg/mL polysorbate 80 | 5.5 |
| | | | dihydrate | | | |
| (9) | 160.2 mg/mL | 5 mmol/L citrate | 170 mmol/L trehalose | 34 mmol/L L-lysine | 0.2 mg/mL polysorbate 80 | 5.6 |
| | | | dihydrate | | | |
| (10) | 144.3 mg/mL | 5 mmol/L citrate | 170 mmol/L trehalose | 57 mmol/L L-proline | 0.2 mg/mL polysorbate 80 | 5.5 |
| | | | dihydrate | | | |
| (11) | 148.8 mg/mL | 5 mmol/L citrate | 170 mmol/L trehalose | 57 mmol/L L-glycine | 0.2 mg/mL polysorbate 80 | 5.4 |
| | | | dihydrate | | | |
| (12) | 158.8 mg/mL | 5 mmol/L citrate | 170 mmol/L trehalose | 57 mmol/L L- | 0.2 mg/mL polysorbate 80 | 5.5 |
| | | | dihydrate | phenylalanine | | |
| (13) | 159.2 mg/mL | 5 mmol/L citrate | 150 mmol/L trehalose | 40 mmol/L sodium | 0.2 mg/mL polysorbate 80 | 5.5 |
| | | | dihydrate | chloride | | |
| (14) | 158.0 mg/mL | 5 mmol/L citrate | 170 mmol/L trehalose | 34 mmol/L L-arginine²⁾ | 0.2 mg/mL polysorbate 80 | 5.5 |
| | | | dihydrate | | | |
| (15) | 170.1 mg/mL | 5 mmol/L citrate | 120 mmol/L trehalose | 60 mmol/L L-arginine²⁾ | 0.2 mg/mL polysorbate 80 | 5.6 |
| | | | dihydrate | | | |
| (16) | 158.1 mg/mL | 5 mmol/L citrate | 195 mmol/L trehalose | 20 mmol/L sodium | 0.2 mg/mL polysorbate 80 | 5.5 |
| | | | dihydrate | chloride | | |

¹⁾pH was adjusted if required by addition of NaOH/ HCl before concentration step
²⁾pH was adjusted if required by addition of NaOH/ phosphoric acid

TABLE 3
Accelerated ageing of pembrolizumab formulations

| Stress Type/Time Point | t0 | 1 week | 2 weeks |
|---|---|---|---|
| 40° C. ± 2° C./75% RH ± 5% RH | X | X | X |

| | |
|---|---|
| Mechanical stressing (up to | 2 conditions (orbital shaking and |
| 24 h) | overhead rotation) |
| Freeze/thaw (−80° C./+25° C.) | 5 cycles |

Samples from all pull points were analyzed by size exclusion chromatography (SE-HPLC) for the presence of high molecular weight species (HMWS) and by ion exchange chromatography (IEX-HPLC) to detect modifications leading to charge heterogeneities. Also, at each pull point the solutions were controlled for visual appearance, for the presence of subvisible particles by dynamic light scattering and pH.

All formulations prepared according to Table 2 were analyzed for their protein content as well as for dynamic viscosity and osmolality before starting the stability study (t0).

b. Protein Content, Osmolality, PH, Visual Inspection of Prepared Formulations

Methods were performed as described in example 1.

All samples prepared met the acceptance criteria for formulations according to Table 2 in protein content (145 mg/mL to 170 mg/mL pembrolizumab), pH (target±0.2) and osmolality (240-320 mOsmol/kg). All these parameters stayed constant during the storage for 2 weeks at 40° C./75% as well as after performing freeze/thaw cycles and mechanical stress. No visible particles or changes in color were detected in all samples.

c. Analysis of Shear Viscosity

All formulations prepared according to Table 2 were analyzed for their dynamic viscosity after preparation as described in example 1.

All samples were analyzed as single measurements and results are shown in Table 4.

Formulations (3), comprising 80 mmol/L trehalose dihydrate and 80 mmol L-arginine as stabilizer, and (4), comprising 120 mmol/L L-arginine as stabilizer, showed lowest dynamic viscosities of 9.4 and 9.0 mPa*s respectively, compared to formulation (1), which is based on trehalose dihydrate without additional stabilizer/viscosity reducing agent.

It was also demonstrated that lysine effectively reduces shear viscosity when added as excipient. In formulation (7), containing 80 mmol/L L-lysine and 80 mmol/L trehalose dihydrate, shear viscosity was dramatically lowered when compared to formulations without addition of a viscosity reducing agent (1).

Finally, both amino acids L-arginine and L-lysine were identified as best viscosity reducing agents when added in concentrations of at least 34 mmol/L in the presence of trehalose. Surprisingly it was shown that also formulations containing only the amino acid L-arginine without any sugar have the lowest viscosity.

All other amino acids only showed no or low effect on viscosity reduction, or even showed an increase of viscosity (formulation (12), comprising L-phenylalanine in combination with trehalose dihydrate).

TABLE 4
Viscosity measurement of high concentration pembrolizumab

| Formulation | Shear viscosity [mPa*sec) |
|---|---|
| (1) | 17.8 |
| (2) | 15.1 |
| (3) | 9.4 |
| (4) | 9.0 |
| (5) | 15.7 |
| (6) | 16.8 |
| (7) | 11.8 |
| (8) | 14.6 |
| (9) | 15.3 |
| (10) | 15.2 |
| (11) | 13.5 |
| (12) | 32.3 |
| (13) | 15.0 |
| (14) | 13.4 |
| (15) | 15.0 |
| (16) | 20.2 |

d. Analysis of High Molecular Weight Species (HMWS) by SE-HPLC

The method was performed as described in example 1.

After storage for two weeks at 40° C./75% RH, HMWS levels slightly increased by 0.4% to 1.0% compared to t0 in all formulations tested (see Table 5).

Best stabilization effects against aggregation were achieved when using trehalose dihydrate without any additional stabilizer as shown in formulation (1), but also partially replacing trehalose dihydrate by amino acids L-arginine, L-lysine and mixtures thereof led to identical or just slightly increased generation of HMWS. Surprisingly, also a formulations in which trehalose was completely replaced with L-arginine (here: 120 mmol/L arginine (formulation (4)) was identified as a feasible formulation to prevent pembrolizumab from aggregation.

Also, the addition of L-phenylalanine to trehalose showed good results at accelerated storage conditions, whereas all other amino acids as well as the addition of sodium chloride led to worse results (formulations (13) and (16)).

All tested formulations remained stable after multiple freeze/thaw cycles as well as after performing physical stress conditions orbital shaking and overhead rotation.

TABLE 5
Generation of HMWS during stability program

| | | | | Δ | Δ | |
|---|---|---|---|---|---|---|
| | | Δ | Δ | HMWS | HMWS | |
| | | HMWS | HMWS | [%] | [%] | Δ |
| | | [%] | [%] | 24 h | 24 h | HMWS |
| Formu- | HMWS | 1 w | 2 w | orbital | overhead | [%] 5x |
| lation | [%] T0 | 40° C. | 40° C. | shaking | rotation | freeze/thaw |
| (1) | 1.0 | 0.5 | 0.4 | 0.0 | 0.0 | −0.1 |
| (2) | 0.9 | 0.5 | 0.4 | 0.0 | 0.0 | −0.1 |
| (3) | 0.8 | 0.5 | 0.5 | 0.1 | 0.0 | −0.1 |
| (4) | 0.7 | 0.5 | 0.6 | 0.1 | 0.1 | 0.0 |
| (5) | 0.8 | 0.5 | 0.6 | 0.1 | 0.1 | 0.0 |
| (6) | 0.9 | 0.5 | 0.6 | 0.0 | 0.0 | −0.1 |
| (7) | 0.7 | 0.5 | 0.7 | 0.1 | 0.1 | 0.0 |
| (8) | 0.8 | 0.5 | 0.7 | 0.1 | 0.1 | −0.1 |
| (9) | 0.8 | 0.6 | 0.8 | 0.1 | 0.1 | 0.0 |
| (10) | 0.9 | 0.6 | 0.8 | 0.0 | 0.1 | −0.1 |
| (11) | 0.8 | 0.6 | 0.8 | 0.0 | 0.0 | −0.1 |
| (12) | 0.8 | 0.5 | 0.7 | 0.0 | 0.0 | −0.1 |
| (13) | 0.9 | 0.7 | 1.0 | 0.1 | 0.1 | −0.1 |
| (14) | 0.8 | 0.6 | 0.8 | 0.0 | 0.1 | −0.1 |
| (15) | 0.8 | 0.6 | 0.8 | 0.0 | 0.1 | 0.0 |
| (16) | 0.9 | 0.7 | 1.0 | 0.1 | 0.1 | −0.1 |

e. Detection of Acidic and Basic Species by IEX-HPLC

The method was performed as described in example 1.

Table 6 summarizes the percentage of the peak area for the acidic species in relation to the total peak area of the eluted species analyzed in IEX-HPLC for the formulations shown in Table 2.

After storage for two weeks at 40° C./75% RH, the acidic species levels increased by 3.1% to 5.1% compared to t0.

Best results were obtained for formulations containing the amino acids L-arginine or L-lysine, as only stabilizer (4), in combinations thereof (5) or when formulated in combination with trehalose dihydrate (formulations (2), (3), (6), (7), (8) and (9)). Interestingly, a titration of L-arginine with phosphoric acid (formulations (14) and (15), leading to L-arginine phosphate instead of L-arginine HCl) led to worse results. Also, formulations containing L-proline, L-glycine and L-phenylalanine in combination with trehalose dihydrate showed worse results, here a higher increase of acidic species.

In formulations combining trehalose dihydrate and sodium chloride (formulations (13) and (16)) the lowest increase of acidic species was detected. Here the addition of sodium chloride to trehalose dihydrate improved stability when compared to a formulation containing only trehalose dihydrate as stabilizer (1).

All tested formulations remained stable after multiple freeze/thaw cycles as well as after performing physical stress conditions orbital shaking and overhead rotation.

In summary, the addition of L-lysine, L-arginine and sodium chloride to trehalose dihydrate improved chemical stability against generation of acidic species compared to a formulation based on trehalose dihydrate without these additional excipients (1). Interestingly, also here a formulation based on L-arginine, without the presence of trehalose dihydrate, is a preferred option as it led to best stabilization effects.

Table 7 summarizes the percentage of the peak area for the basic species in relation to the total peak area of the eluted species analyzed in IEX-HPLC for the formulations shown in Table 2.

After storage for two weeks at 40° C./75% RH, the basic species levels decrease by 2.6% to 5.4% compared to t0.

Best results for stabilizing basic species against decrease were obtained for formulations containing the amino acids L-arginine or L-lysine, either alone (4)(5), or when each of them was formulated in combination with trehalose dihydrate (formulations (2), (3), (6), (7), (8) and (9)). One formulation comprising 80 mmol/L L-lysine with 80 mmol/L trehalose dihydrate (7) showed the best stabilizing effects, slightly superior to other tested formulations.

Interestingly, a titration of L-arginine with phosphoric acid (formulations (14) and (15), leading to L-arginine phosphate instead of L-arginine HCl) led to worse results. Also, formulations containing L-proline, L-glycine and L-phenylalanine in combination with trehalose dihydrate showed worse results, here a higher decrease of basic species.

All tested formulations remained stable after multiple freeze/thaw cycles as well as after performing physical stress conditions orbital shaking and overhead rotation.

In summary, the addition of L-lysine and/or L-arginine to trehalose dihydrate improved chemical stability against decrease of basic species compared to a formulation based on trehalose dihydrate without these additional excipients. The use of L-lysine was shown to be slightly beneficial compared to L-arginine in these experiments.

Interestingly, also here a formulation based on L-arginine, without the presence of trehalose dihydrate, is one of the preferred options as it led to very promising stabilization effects.

TABLE 6
Generation of acidic species analyzed
by IEX-HPLC during stability program

| | | | | Acidic | Acidic | |
|---|---|---|---|---|---|---|
| | | | | species | species | |
| | | Acidic | Acidic | [%] | [%] | Acidic |
| | Acidic | species | species | 24 h | 24 h | species |
| Formu- | species | [%] 1 w | [%] 2 w | orbital | overhead | [%] 5x |
| lation | [%] T0 | 40° C. | 40° C. | shaking | rotation | freeze/thaw |
| (1) | 18.0 | 1.8 | 3.9 | 0.2 | 0.3 | 0.0 |
| (2) | 18.0 | 1.6 | 3.4 | 0.1 | 0.1 | 0.1 |
| (3) | 18.2 | 1.4 | 3.2 | 0.0 | 0.1 | 0.0 |
| (4) | 18.0 | 1.4 | 3.2 | 0.1 | 0.1 | −0.1 |
| (5) | 18.0 | 1.5 | 3.3 | 0.1 | 0.1 | 0.0 |
| (6) | 18.2 | 1.4 | 3.2 | 0.1 | 0.0 | 0.0 |
| (7) | 18.1 | 1.4 | 3.1 | 0.1 | 0.0 | 0.0 |
| (8) | 18.3 | 1.4 | 3.5 | 0.0 | 0.0 | −0.2 |
| (9) | 18.2 | 1.6 | 3.4 | 0.1 | 0.1 | 0.0 |
| (10) | 18.4 | 2.0 | 4.2 | 0.1 | 0.0 | −0.1 |
| (11) | 18.3 | 2.0 | 4.3 | 0.1 | 0.1 | 0.0 |
| (12) | 18.6 | 2.1 | 4.5 | 0.1 | 0.1 | 0.0 |
| (13) | 18.3 | 1.6 | 3.3 | 0.0 | 0.1 | −0.1 |
| (14) | 18.3 | 2.5 | 4.8 | 0.2 | 0.1 | −0.1 |
| (15) | 18.3 | 2.7 | 5.1 | 0.1 | 0.0 | −0.1 |
| (16) | 18.3 | 1.6 | 3.2 | 0.0 | 0.0 | −0.1 |

TABLE 7
Generation of basic species analyzed
by IEX-HPLC during stability program

| | | | | Basic | Basic | |
|---|---|---|---|---|---|---|
| | | | | species | species | |
| | | Basic | Basic | [%] | [%] | Basic |
| | Basic | species | species | 24 h | 24 h | species |
| Formu- | species | [%] 1 w | [%] 2 w | orbital | overhead | [%] 5x |
| lation | [%] T0 | 40° C. | 40° C. | shaking | rotation | freeze/thaw |
| (1) | 27.4 | −1.3 | −3.6 | −0.2 | −0.3 | −0.1 |
| (2) | 27.2 | −1.1 | −3.4 | −0.1 | −0.2 | 0.0 |
| (3) | 27.0 | −1.0 | −3.0 | −0.1 | −0.1 | 0.0 |
| (4) | 27.3 | −0.8 | −2.9 | −0.1 | −0.1 | 0.0 |
| (5) | 27.3 | −0.9 | −2.8 | −0.1 | −0.1 | −0.1 |
| (6) | 27.2 | −0.9 | −2.9 | −0.1 | −0.2 | −0.1 |
| (7) | 27.1 | −0.8 | −2.6 | −0.1 | −0.1 | −0.1 |
| (8) | 27.0 | −1.1 | −3.4 | −0.1 | 0.0 | 0.1 |
| (9) | 27.0 | −1.1 | −3.2 | −0.3 | −0.1 | −0.1 |
| (10) | 26.9 | −1.7 | −4.1 | −0.2 | −0.2 | −0.1 |
| (11) | 27.1 | −1.7 | −4.2 | −0.4 | −0.2 | −0.3 |
| (12) | 26.9 | −1.8 | −4.2 | −0.3 | −0.2 | −0.2 |
| (13) | 27.1 | −1.2 | −3.4 | −0.3 | −0.2 | −0.1 |
| (14) | 26.8 | −3.7 | −5.4 | −0.6 | −0.4 | −0.2 |
| (15) | 26.6 | −3.6 | −5.1 | −0.6 | −0.5 | −0.1 |
| (16) | 26.9 | −1.2 | −3.5 | −0.2 | 0.0 | 0.0 |

f. Summary of Study Results

Based on the results generated with the shown method set some excipients and combinations thereof were identified, which both stabilize pembrolizumab at high concentrations in liquid formulations against physico-chemical degradation and show a positive impact on viscosity, here a reduction of viscosity, to allow the subcutaneous application route for highly concentrated pembrolizumab formulations.

Especially the amino acids L-lysine and L-arginine were identified to have a positive impact on viscosity reduction as well as on stabilizing pembrolizumab against generation of HMWS (analyzed by SE-HPLC) and to stabilize acidic and basic species (analyzed by IEX-HPLC).

Both L-lysine as well as L-arginine were very powerful in optimizing the formulation when combined with trehalose dihydrate, all mixtures were superior compared to formulations containing only trehalose dihydrate (1). Best results were achieved when adding 60 mmol/L L-arginine to 120 mmol/L trehalose dihydrate (2), or 80 mmol/L L-arginine to 80 mmol/L trehalose dihydrate (3) or 80 mmol/L L-lysine to 80 mmol/L trehalose dihydrate (7). When comparing L-arginine (3) with L-lysine (7), the use of L-lysine was superior to arginine in stabilization against decrease of basic species.

Also, formulation (4) comprising 120 mmol/L L-arginine without the presence of trehalose led to comparable excellent physico-chemical results as well as lowest viscosity of all tested formulations, which makes this sugar-free formulation a preferred option to stabilize pembrolizumab at high concentrations.

L-proline, L-phenylalanine and L-glycine did not show any improvement on stability, or even had negative effects (e.g. mixtures of L-phenylalanine with trehalose lead to a high increase of dynamic viscosity).

The addition of sodium chloride showed a positive effect on physico-chemical stabilization of pembrolizumab, but the higher increase in HMWS makes this a non-preferred formulation candidate.

Titrating L-arginine with phosphoric acid, leading to the presence of L-arginine phosphate (14) and (15), showed a negative effect on all physico-chemical parameters, the preferred option is therefore the buffer system L-arginine/L-arginine HCL, which either can be obtained by mixing both components to reach the final pH or titrating L-arginine with HCl.

### Example 3: Buffer Screening and Optimizing Polysorbate 80 Concentration

a. Sample Preparation

A short-term stability study including mechanical stress and freeze/thaw stress was performed in order to screen for potential buffer systems suitable for generating stable formulations of highly concentrated pembrolizumab at pH 5.5.

Several buffer systems providing buffer capacity at pH 5.5 were systematically screened to evaluate their effects on both physico-chemical stabilization as well as viscosity reduction of the solution: citrate, acetate, succinate, adipate, malate and L-histidine. These buffers were tested in two exemplary combinations of excipients based on the outcome of example 1, whereas one formulation consisted of 120 mmol/L trehalose dihydrate, 70 mmol/L L-arginine, 0.2 mg/mL polysorbate 80, pH 5.5, the other one consisted of 150 mmol/L L-arginine and 0.2 mg/mL polysorbate 80, pH 5.5. All formulations contained pembrolizumab at a concentration of 165 mg/mL±10%.

In addition, a screening of polysorbate 80 was performed to evaluate effects of polysorbate 80 concentration on physico-chemical stability. Solutions were tested providing a polysorbate 80 concentration range from 0.1 mg/mL to 0.4 mg/mL in an exemplary formulation of 165 mg/mL pembrolizumab, 5 mmol/L citrate, 120 mmol/L trehalose dihydrate, 70 mmol/L L-arginine and 0.2 mg/mL polysorbate 80, pH 5.5.

Pembrolizumab with a concentration of about 28 mg/mL, formulated in 10 mmol/L citrate, 300 mmol/L L-arginine at pH 5.5 (surfactant free) was used as starting material and 14 new formulations as listed in Table 8 were created by the use of a Big Tuna automated buffer exchange system (Unchained Labs, Pleasanton, CA) with a 30 kDa MWCO filter made of polypropylene with regenerated cellulose and providing at least 96% formulation exchange.

After that pembrolizumab concentration was increased and solutions were spiked with polysorbate 80 to the target concentration.

The formulations shown in Table 8 were initially analyzed for protein concentration by UV-VIS spectroscopy at 280 nm and correct pH adjustment before starting an accelerated ageing stability program. Storage conditions are shown in Table 9. In brief, samples were placed on storage for up to 4 weeks at 40° C./75% relative humidity (RH). In addition, samples were subjected to two conditions of mechanical stressing (overhead rotation, orbital shaking) as well as five freeze/thaw cycles (−80° C./+25° C.).

TABLE 8
Detailed information of formulations prepared within this study

| | | | Stabilizer | Stabilizer | | |
|---|---|---|---|---|---|---|
| | | Buffer | 1/tonicity | 2/tonicity | | |
| No. | Pembrolizumab | system ¹⁾ | agent 1 | agent 2²⁾ | Surfactant | pH |
| (1) | 165 mg/ml | 5 mmol/L | 120 mmol/L | 70 mmol/L | 0.2 mg/mL | 5.5 |
| | | citrate | trehalose | L-arginine | polysorbate 80 | |
| | | | dihydrate | | | |
| (2) | 165 mg/mL | 5 mmol/L | 120 mmol/L | 70 mmol/L | 0.1 mg/mL | 5.5 |
| | | citrate | trehalose | L-arginine | polysorbate 80 | |
| | | | dihydrate | | | |
| (3) | 165 mg/ml | 5 mmol/L | 120 mmol/L | 70 mmol/L | 0.4 mg/mL | 5.5 |
| | | citrate | trehalose | L-arginine | polysorbate 80 | |
| | | | dihydrate | | | |
| (4) | 165 mg/ml | 10 mmol/L | 120 mmol/L | 70 mmol/L | 0.2 mg/mL | 5.5 |
| | | acetate | trehalose | L-arginine | polysorbate 80 | |
| | | | dihydrate | | | |
| (5) | 165 mg/mL | 10 mmol/L | 120 mmol/L | 70 mmol/L | 0.2 mg/mL | 5.5 |
| | | succinate | trehalose | L-arginine | polysorbate 80 | |
| | | | dihydrate | | | |
| (6) | 165 mg/mL | 10 mmol/L | 120 mmol/L | 70 mmol/L | 0.2 mg/mL | 5.5 |
| | | adipate | trehalose | L-arginine | polysorbate 80 | |
| | | | dihydrate | | | |
| (7) | 165 mg/mL | 10 mmol/L | 120 mmol/L | 70 mmol/L | 0.2 mg/mL | 5.5 |
| | | malate | trehalose | L-arginine | polysorbate 80 | |
| | | | dihydrate | | | |
| (8) | 165 mg/mL | 5 mmol/L | n.a. | 150 mmol/L | 0.2 mg/mL | 5.5 |
| | | citrate | | L-arginine | polysorbate 80 | |
| (9) | 165 mg/mL | 10 mmol/L | n.a. | 150 mmol/L | 0.2 mg/mL | 5.5 |
| | | acetate | | L-arginine | polysorbate 80 | |
| (10) | 165 mg/mL | 10 mmol/L | n.a. | 150 mmol/L | 0.2 mg/mL | 5.5 |
| | | succinate | | L-arginine | polysorbate 80 | |
| (11) | 165 mg/ml | 10 mmol/L | n.a. | 150 mmol/L | 0.2 mg/mL | 5.5 |
| | | adipate | | L-arginine | polysorbate 80 | |
| (12) | 165 mg/mL | 10 mmol/L | n.a. | 150 mmol/L | 0.2 mg/mL | 5.5 |
| | | malate | | L-arginine | polysorbate 80 | |

¹⁾pH was adjusted if required by addition of NaOH/HCl before concentration step
²⁾Arginine with its pka values 12.48, 9.04 and 2.17 doesn't provide buffering capacity at pH 5.5, that's why it's classified as stabilizer and not as buffering agent

TABLE 9
Accelerated aging of pembrolizumab formulations

| Stress Type/Time | t0 | 1 week | 2 weeks | 4 weeks |
|---|---|---|---|---|
| Point | X | X | X | X |
| 40° C. ± 2° C./75% | | | | |
| RH ± 5% RH | | | | |
| Mechanical | | 2 conditions (orbital shaking | | |
| stressing (up | | and overhead rotation) | | |
| to 24 h) | | | | |
| Freeze/ thaw | | 5 cycles | | |
| (−80° C./+25° C.) | | | | |

Samples from all pull points were analyzed by size exclusion chromatography (SE-HPLC) for the presence of high molecular weight species (HMWS) and by ion exchange chromatography (IEX-HPLC) to detect modifications leading to charge heterogeneities. Also, at each pull point the solutions were controlled for visual appearance, for the presence of subvisible particles by flow imaging microscopy and pH.
b. Analysis of Protein Content by UV-VIS and Determination of pH and Osmolality

All formulations prepared according to Table 8 were analyzed for their protein content, dissociation constant Kd as well as for pH and osmolality before starting the stability study (t0). All samples met the acceptance criteria for all formulations listed in Table 8 in protein content (165 mg/mL pembrolizumab±10%), pH (target±0.2) and osmolality (300 mOsm/kg±20%).

No visible particles or changes in color were detected in all samples.

c. Analysis of Shear Viscosity

All formulations prepared according to Table 8 were analyzed for their dynamic viscosity after preparation. The method was performed as described in example 1.

Shear viscosity measurements demonstrate that formulations (8) to (12), all based on 150 mmol/L L-arginine, have much lower viscosities than formulations (1) to (7), all containing a sugar, here 120 mmol/L trehalose dihydrate in combination with 70 mmol/L L-arginine. Out of scope of this ranking is formulation (2) due to higher content of pembrolizumab, as the concentration of pembrolizumab has also a strong impact on viscosity and is overlapping the effect of the excipient screening.

Comparing the shear viscosity in formulations (8) to (12), only a slight tendency with a higher value for malate was detected, a very slight improvement could be determined with the use of succinate (10), all other buffer systems were quantified in a similar range.

Comparing the formulations (1) to (7), a slight improvement with regard to lower shear viscosity could be achieved using adipate (6) as buffer system if formulated with 120 mmol/L trehalose dihydrate and 70 mmol/L L-arginine.

Finally, formulations of 165 mg/ml pembrolizumab, formulated with 150 mmol/L L-arginine, 0.2 mg/mL polysorbate 80 have low shear viscosities, independent of the used buffering system.

TABLE 10
Viscosity measurements and concentrations of pembrolizumab

| Formulation | Shear viscosity [mPa*sec) | Pembrolizumab [mg/mL] |
|---|---|---|
| (1) | 19.2 | 171.1 |
| (2) | 27.2 | 183.7 |
| (3) | 21.8 | 172.2 |
| (4) | 18.2 | 164.2 |
| (5) | 19.3 | 166.4 |
| (6) | 15.8 | 159.7 |
| (7) | 19.9 | 169.0 |
| (8) | 14.2 | 168.6 |
| (9) | 14.3 | 167.8 |
| (10) | 13.1 | 164.6 |
| (11) | 14.0 | 167.2 |
| (12) | 15.2 | 167.5 |

d. Analysis of Colloidal Stability (Kd) by Dynamic Light Scattering

Samples comprising higher negative values for Kd indicate an increase of protein-protein attractive forces. Samples (9) to (12), all comprising 150 mmol/L L-arginine without the presence of trehalose dihydrate, buffered either by acetate, succinate, adipate or malate, showed much better results, indicated by higher Kd (absolute number of negative values is smaller), compared to all other tested formulations. For these formulations (9) to (12) attractive forces were minimized compared to samples comprising a sugar, a favorized attribute for stabilization of pembrolizumab.

TABLE 11
Kd measurements of highly concentrated
formulations of pembrolizumab

| Formulation | Kd viscosity corrected * 1000 |
|---|---|
| (1) | −10.2 |
| (2) | −11.0 |
| (3) | −10.1 |
| (4) | −10.1 |
| (5) | −8.1 |
| (6) | −12.1 |
| (7) | −7.6 |
| (8) | −8.1 |
| (9) | −4.4 |
| (10) | −5.1 |
| (11) | −4.9 |
| (12) | −5.3 |

e. Analysis of High Molecular Weight Species (HMWS) by SE-HPLC

The method was performed as described in example 1.

After storage for four weeks at 40° C./75% RH, HMWS levels increased slightly by 1.2% to 1.4% compared to t0 in all formulations tested (see Table 12), low values for these accelerated conditions of storage.

All tested formulations were evaluated in a similar range regarding the generation of HMWS, no clear trend was detectable after analyzing the pull points. Slightly worse results were detected when using a malate buffered formulation with 120 mmol/L trehalose dihydrate and 70 mmol/L L-arginine (7).

Finally, all tested buffers seem to be suitable to generate promising formulations of pembrolizumab at 165 mg/mL with regard to stabilization against high molecular weight species.

No impact of polysorbate 80 in the tested range could be detected in SE-HPLC, the formulations behave identical in the range of 0.1 mg/mL to 0.4 mg/mL polysorbate 80 in a formulation comprising 165 mg/mL pembrolizumab with 5 mmol/L citrate, 120 mmol/L trehalose dihydrate and 70 mmol/L L-arginine at pH 5.5.

All tested formulations remained stable after multiple freeze/thaw cycles as well as after performing physical stress conditions orbital shaking and overhead rotation.

TABLE 12
Generation of HMWS during stability program

| | HMWS | Δ HMWS | Δ HMWS | Δ HMWS | Δ HMWS | Δ HMWS | |
|---|---|---|---|---|---|---|---|
| | [%] | [%] 1 w | [%] 2 w | [%] 4 w | [%] 24 h | [%] 24 h | Δ HMWS [%] 5x |
| Formulation | T0 | 40° C. | 40° C. | 40° C. | orbital shaking | overhead rotation | freeze/thaw |
| (1) | 0.9 | 0.7 | 0.9 | 1.3 | 0.1 | 0.1 | 0.0 |
| (2) | 1.0 | 0.7 | 1.0 | 1.3 | 0.1 | 0.1 | 0.1 |
| (3) | 1.0 | 0.7 | 1.0 | 1.3 | 0.1 | 0.1 | 0.1 |
| (4) | 0.9 | 0.7 | 1.0 | 1.3 | 0.2 | 0.1 | 0.1 |
| (5) | 0.9 | 0.7 | 1.0 | 1.2 | 0.1 | 0.1 | 0.0 |
| (6) | 1.0 | 0.7 | 0.9 | 1.2 | 0.2 | 0.0 | 0.0 |
| (7) | 0.8 | 0.6 | 1.1 | 1.4 | 0.2 | 0.1 | 0.1 |
| (8) | 0.8 | 0.7 | 0.9 | 1.2 | 0.1 | 0.3 | 0.1 |
| (9) | 0.9 | 0.6 | 0.9 | 1.2 | 0.1 | 0.0 | 0.0 |
| (10) | 0.7 | 0.7 | 1.0 | 1.3 | 0.1 | 0.1 | 0.1 |
| (11) | 0.7 | 0.7 | 0.9 | 1.2 | 0.1 | 0.1 | 0.1 |
| (12) | 0.7 | 0.7 | 0.9 | 1.3 | 0.1 | 0.1 | 0.1 |

f. Detection of Acidic and Basic Species by IEX-HPLC

The method was performed as described in example 1.

Table 13 summarizes the percentage of the peak area for the acidic species in relation to the total peak area of the eluted species analyzed in IEX-HPLC for the formulations shown in Table 8.

After storage for four weeks at 40° C./75% RH, the acidic species levels increased by 13.1% to 14.0% compared to t0.

All tested buffers seem to be suitable to generate promising formulations of pembrolizumab at 165 mg/mL regarding stabilization against generation of acidic species tested in the different exemplary formulations.

When using 150 mmol/L L-arginine (formulations (8) to (12)), all tested buffer systems work similarly well.

No impact of polysorbate 80 regarding stabilization against the increase of acidic species could be detected in IEX-HPLC, the formulations behave identical in the range of 0.1 mg/mL to 0.4 mg/mL polysorbate 80 in a formulation comprising 165 mg/mL pembrolizumab with 5 mmol/L citrate, 120 mmol/L trehalose dihydrate and 70 mmol/L L-arginine at pH 5.5.

All tested formulations remained stable after multiple freeze/thaw cycles as well as after applying physical stress conditions orbital shaking and overhead rotation.

Table 14 summarizes the percentage of the peak area for the basic species in relation to the total peak area of the eluted species analyzed in IEX-HPLC for the formulations shown in Table 8.

All tested buffers seem to be suitable to generate promising formulations of pembrolizumab at 165 mg/mL in a comparable way regarding stabilization against loss of basic species tested in the different exemplary formulations.

When using 150 mmol/L L-arginine (formulations (8) to (12)), all tested buffer systems work similarly well, using acetic acid seems to be just slightly superior in this group compared to the others.

No impact of polysorbate 80 in stabilizing basic species could be detected in IEX-HPLC, the formulations behave identical in the range of 0.1 mg/mL to 0.4 mg/mL polysorbate 80 in a formulation comprising 165 mg/mL pembrolizumab with 5 mmol/L citrate, 120 mmol/L trehalose dihydrate and 70 mmol/L L-arginine at pH 5.5.

All tested formulations remained stable after multiple freeze/thaw cycles as well as after applying physical stress conditions orbital shaking and overhead rotation.

TABLE 13
Generation of acidic species analyzed by IEX-HPLC during stability program

| | | Acidic species | Acidic species | Acidic species | Acidic species | Acidic species | Acidic species |
|---|---|---|---|---|---|---|---|
| | Acidic species | [%] 1 w | [%] 2 w | [%] 4 w | [%] 24 h | [%] 24 h | [%] 5x |
| Formulation | [%] T0 | 40° C. | 40° C. | 40° C. | orbital shaking | overhead rotation | freeze/thaw |
| (1) | 20.2 | 1.6 | 3.6 | 13.4 | 0.1 | −0.1 | 0.1 |
| (2) | 20.3 | 1.4 | 3.4 | 13.1 | 0.0 | −0.1 | 0.0 |
| (3) | 20.1 | 1.4 | 3.5 | 13.1 | −0.1 | 0.0 | 0.0 |
| (4) | 20.2 | 1.1 | 3.2 | 13.1 | 0.0 | −0.1 | 0.0 |
| (5) | 20.2 | 1.5 | 3.7 | 14.0 | 0.0 | −0.1 | 0.0 |
| (6) | 20.2 | 1.5 | 3.6 | 13.6 | 0.1 | 0.0 | 0.0 |
| (7) | 20.2 | 1.2 | 3.2 | 13.2 | 0.0 | 0.0 | 0.1 |
| (8) | 20.2 | 1.5 | 3.7 | 13.3 | 0.0 | 0.0 | 0.0 |
| (9) | 20.1 | 1.3 | 3.3 | 13.1 | 0.0 | 0.0 | 0.1 |
| (10) | 20.2 | 1.5 | 3.6 | 13.6 | 0.0 | −0.1 | −0.1 |
| (11) | 20.2 | 1.4 | 3.3 | 13.2 | −0.1 | −0.1 | 0.1 |
| (12) | 20.2 | 1.4 | 3.3 | 13.6 | 0.0 | −0.3 | 0.0 |

TABLE 14
Generation of basic species analyzed by IEX-HPLC during stability program

| | | Basic species | Basic species | Basic species | Basic species | Basic species | Basic species |
|---|---|---|---|---|---|---|---|
| | Basic species | [%] 1 w | [%] 2 w | [%] 4 w | [%] 24 h | [%] 24 h | [%] 5x |
| Formulation | [%] T0 | 40° C. | 40° C. | 40° C. | orbital shaking | overhead rotation | freeze/thaw |
| (1) | 25.1 | −0.9 | −2.0 | −3.7 | −0.2 | 0.0 | −0.1 |
| (2) | 25.0 | −0.9 | −2.0 | −3.6 | −0.1 | 0.0 | −0.1 |
| (3) | 25.1 | −0.8 | −1.9 | −3.5 | −0.1 | 0.0 | 0.0 |
| (4) | 25.3 | −0.5 | −1.6 | −3.1 | 0.0 | −0.1 | −0.1 |
| (5) | 25.2 | −1.0 | −2.4 | −4.0 | −0.1 | −0.1 | −0.1 |
| (6) | 25.2 | −1.1 | −2.2 | −3.8 | −0.2 | −0.1 | −0.1 |
| (7) | 25.2 | −0.6 | −1.6 | −2.9 | 0.0 | 0.0 | 0.0 |
| (8) | 25.2 | −0.8 | −2.0 | −3.9 | 0.0 | 0.0 | −0.1 |
| (9) | 25.1 | −0.4 | −1.4 | −3.4 | 0.0 | 0.0 | 0.0 |
| (10) | 25.2 | −1.0 | −2.2 | −4.2 | −0.1 | −0.1 | −0.1 |
| (11) | 25.0 | −0.9 | −1.9 | −3.9 | 0.1 | 0.0 | 0.0 |
| (12) | 25.2 | −0.6 | −1.6 | −3.7 | −0.1 | 0.1 | 0.0 |

g. Summary of Study Results

Based on the results generated with the shown method sets several buffer systems with buffer capacity at pH 5.5 could be identified, which stabilize on the one hand pembrolizumab in liquid formulations and on the other hand provide stable pH during storage.

The effect of the different buffer systems was evaluated in two different formulations, whereas one formulation was based on a mixture of trehalose dihydrate (120 mmol/L) and L-arginine (70 mmol/L), the other formulation was a sugar-free formulation containing L-arginine at a concentration of 150 mmol/L. All concentrations were adjusted successfully to approximately 165 mg/mL pembrolizumab at pH 5.5 and fulfill requirements for subcutaneous injections of about 300 mOsm/kg.

Especially formulations of 165 mg/mL pembrolizumab in 150 mmol/L L-arginine, 0.2 mg/mL polysorbate 80 (formulations (8) to (12)) were superior to all others when analyzed for shear viscosity. This was independent of the used buffer system. If formulating pembrolizumab with 120 mmol/L trehalose dihydrate and 70 mmol/L L-arginine, the use of adipate (formulation (6)) as buffering system seems to be an option.

Samples (8) to (12), all comprising 150 mmol/L L-arginine without trehalose dihydrate, buffered either by acetate, succinate, adipate or malate, showed much better results in Kd compared to all other tested formulations. For formulations (9) to (12) attractive forces were minimized compared to samples comprising sugars, a favorized attribute for stabilization of pembrolizumab. The best result was achieved when using acetate as buffering agent.

Considering the generation of HMWS (analyzed by SE-HPLC) and stabilization of acidic as well basic species all tested buffers seem to be suitable to generate promising formulations of pembrolizumab at 165 mg/mL, no clear trend was visible.

No impact of polysorbate 80 concentration on all tested quality parameters could be identified, the formulations behave identical in the concentration range of 0.1 mg/mL to 0.4 mg/mL when formulating 165 mg/mL pembrolizumab with 5 mmol/L citrate, 120 mmol/L trehalose dihydrate and 70 mmol/L L-arginine at pH 5.5. This formulation was used as an exemplary formulation, results can be also be assigned to other formulations comprising similar concentrations of pembrolizumab.

All tested formulations remained stable after multiple freeze/thaw cycles as well as after applying physical stress conditions orbital shaking and overhead rotation.

### Example 4: Formulations Selected for Stability Study

Based on the results shown in example 2 and example 3, 9 formulations were selected to be tested in a long-term stability at target storage condition 2° C.-8° C. as well as accelerated conditions 25° C./60% RH for up to 3 months and 40° C./75% RH for up to 1 month.

The same starting material as described in example 3 was used and formulations will be transferred into new formulations by UF/DF. The 9 different formulations are shown in Table 15.

TABLE 15
Detailed information of formulations prepared within this study

| No. | Pembrolizumab | 140 mmol/L | Stabilizer | Surfactant | pH |
|---|---|---|---|---|---|
| (1) | 165 mg/mL | L-lysine | 150 mmol/L | 0.2 mg/mL | 5.5 |
| | | acetate | L-arginine | polysorbate 80 | |
| (2) | 165 mg/mL | 10 mmol/L | 150 mmol/L | 0.2 mg/mL | 5.2 |
| | | acetate | L-arginine | polysorbate 80 | |
| (3) | 165 mg/mL | 10 mmol/L | 150 mmol/L | 0.2 mg/mL | 5.5 |
| | | adipate | L-arginine | polysorbate 80 | |
| (4) | 165 mg/mL | 10 mmol/L | 150 mmol/L | 0.2 mg/mL | 5.5 |
| | | succinate | L-arginine | polysorbate 80 | |
| (5) | 165 mg/mL | 10 mmol/L | 140 mmol/L | 0.2 mg/mL | 5.5 |
| | | acetate | L-lysine | polysorbate 80 | |
| (6) | 165 mg/mL | 10 mmol/L | 140 mmol/L | 0.2 mg/mL | 5.2 |
| | | acetate | L-lysine | polysorbate 80 | |
| (7) | 165 mg/mL | 10 mmol/L | 140 mmol/L | 0.2 mg/mL | 5.5 |
| | | adipate | L-lysine | polysorbate 80 | |
| (8) | 165 mg/mL | 10 mmol/L | 140 mmol/L | 0.2 mg/mL | 5.5 |
| | | succinate | L-lysine | polysorbate 80 | |
| (9) | 165 mg/mL | 10 mmol/L | 70 mmol/L | 0.2 mg/mL | 5.5 |
| | | adipate | L-lysine/120 | polysorbate 80 | |
| | | | mmol/L | | |
| | | | trehalose | | |
| | | | dihydrate | | |

Samples will be stored for up to 24 months according to Table 16. In addition, samples will be subjected to two conditions of mechanical stressing as well as five freeze/thaw cycles.

TABLE 16
Storage stability study of Pembrolizumab

| | Time Point (months) | | | | | | | | | |
|---|---|---|---|---|---|---|---|---|---|---|
| Stress Type | t0 | 0.5 | 1 | 2 | 3 | 6 | 9 | 12 | 18 | 24 |
| 5° C. | X | | X | | X | X | X | X | X | X |
| 25° C. ± 2° C. / | | | X | X | X | | | | | |
| 60% RH ± 5% RH | | | | | | | | | | |
| 40° C. ± 2° C. / | | X | X | | | | | | | |
| 75% RH ± 5% RH | | | | | | | | | | |

| | |
|---|---|
| Mechanical stressing | 2 conditions (orbital shaking |
| (up to 24 h) | and overhead rotation) |
| Freeze/ thaw | 5 cycles |
| (−80° C. / +25° C.) | |

Protein stability will be determined by size exclusion chromatography (SE-HPLC) for the presence of high molecular weight species (HMWS) and by non-reducing SDS-cGE for the presence of low molecular weight species (LMWS) and HMWS. Chemical modifications like glycation, oxidation and deamidation will be quantified by LC-ESI-MS and -MS/MS in reduced peptide mapping, oxidation will also be monitored by HIC chromatography. Ion exchange chromatography (IEX-HPLC) as well as imaged capillary isoelectric focusing (icIEF) will be used to detect modifications leading to charge heterogeneities. DLS will be analyzed, also to calculate Kd values for charactering protein—protein interactions. In addition, samples will be analyzed for appearance, viscosity, turbidity, sub-visible particle content and particle size. The protein concentration of the samples will be determined by UV-VIS spectroscopy. The pH will also be monitored during the stability study.

### Example 5: Stability Study of Selected Formulations for High Concentrated Pembrolizumab

Based on the results shown in example 2 and example 3, 5 formulations were selected to be tested in a long-term stability study at target storage condition 2° C.-8° C. for up to 9 months as well as accelerated conditions 25° C./60% RH for up to 3 months and 40° C./75% RH for up to 1 month.

Pembrolizumab with a concentration of about 33 mg/mL, formulated in 10 mmol/L citrate, 300 mmol/L L-arginine at pH 5.5 (surfactant free) was used as starting material, bulk filtered using Sartorius Sartopore 2 process filter (0.45+0.2 μm PES membrane, cat. #5441307H4G-00) and 5 new formulations as listed in Table 17 were created. The buffer exchange and concentration of pembrolizumab was conducted using tangential flow filtration on a Sartorius Ambr Crossflow device and Sartocon Slice 50 filter cassettes (30 kDa, cat. #3D91465950MLPU). The buffer exchange and concentration process were performed sequentially as initial concentration—diafiltration—final concentration process: At first, the drug substance was concentrated to reduce the process volume, followed by dialysis with 5 diafiltration volumes for continuous buffer exchange, resulting in a theoretical buffer exchange of more than 99%. Finally, the drug substance was concentrated to its target concentration in the final formulations and the solutions were sterile filtered using a 0.22 μm vacuum filter (Steriflip Sterile 50 mL PES Disposable Vacuum Filter System from Merck, cat #SCGP00525) according to manufacturer's instruction. After that the solutions were spiked with sterile polysorbate 80 solution leading to the target concentration and filled into sterile, depyrogenated, particle-free 2R vials (Adelphi, cat #1404.6.1.1) under laminar air flow conditions. The filled vials were closed with sterile 13 mm injection stoppers (Adelphi, cat. #1625.6.1.1) and crimped with aluminum caps (Adelphi, cat #CETW13). All vials were filled with 500 μl of solution. The formulations shown in Table 17 were initially analyzed for protein concentration by UV-VIS spectroscopy at 280 nm and correct pH adjustment before starting the storage for the stability study.

TABLE 17
Detailed information of formulations prepared within this study

| | | Buffer | | | |
|---|---|---|---|---|---|
| No. | Pembrolizumab | system | Stabilizer | Surfactant | pH |
| (1) | 165 mg/mL | 10 mmol/L | 150 mmol/L | 0.2 mg/mL | 5.5 |
| | | acetate | L-arginine | polysorbate 80 | |
| (2) | 165 mg/mL | 10 mmol/L | 150 mmol/L | 0.2 mg/mL | 5.2 |
| | | acetate | L-arginine | polysorbate 80 | |
| (3) | 165 mg/mL | 10 mmol/L | 150 mmol/L | 0.2 mg/mL | 5.5 |
| | | adipate | L-arginine | polysorbate 80 | |
| (4) | 165 mg/mL | 10 mmol/L | 140 mmol/L | 0.2 mg/mL | 5.5 |
| | | acetate | L-lysine | polysorbate 80 | |
| (5) | 165 mg/mL | 10 mmol/L | 70 mmol/L | 0.2 mg/mL | 5.5 |
| | | adipate | L-lysine/120 | polysorbate 80 | |
| | | | mmol/L | | |
| | | | trehalose | | |
| | | | dihydrate | | |

Storage conditions are shown in Table 18. Sample storage will be continued for up to 24 months as described in Table 18.

TABLE 18
Storage stability study of Pembrolizumab

| | Time Point (months) | | | | | | | | | |
|---|---|---|---|---|---|---|---|---|---|---|
| Stress Type | t0 | 0.5 | 1 | 2 | 3 | 6 | 9 | 12 | 18 | 24 |
| 5° C. | X | | X | | X | X | X | X | X | X |
| 25° C. ± 2° C. / | | | X | X | X | | | | | |
| 60% RH ± 5% RH | | | | | | | | | | |
| 40° C. ± 2° C. / | | X | X | | | | | | | |
| 75% RH ± 5% RH | | | | | | | | | | |

| | |
|---|---|
| Mechanical stressing | 2 conditions (orbital shaking at 400 rpm |
| (24 h) | and overhead rotation at 30 rpm) |
| Freeze/ thaw | 5 cycles |
| (−80° C. / +25° C.) | |

Protein stability was determined by size exclusion chromatography (SE-HPLC) for the presence of high molecular weight species (HMWS) and by non-reducing SDS-cGE for the presence of low molecular weight species (LMWS). Oxidation was quantified by using HIC chromatography. Ion exchange chromatography (IEX-HPLC) as well as imaged capillary isoelectric focusing (icIEF) were used to detect modifications leading to charge heterogeneities. DLS was used for characterizing samples for the presence of aggregates, and also to calculate Kd values for characterizing protein—protein interactions. In addition, samples were analyzed for appearance, viscosity, turbidity, sub-visible particle content and particle size. The protein concentration of the samples was determined by UV-VIS spectroscopy. Also, pH and dynamic viscosity was analyzed during the stability study. If not mentioned below, all methods are described and were performed according to example 1.

a. Analysis of Protein Content by UV-VIS, Determination of PH, Visible Particles, Turbidity, Subvisible Particles and Osmolality

All prepared formulations according to Table 17 were analyzed for their protein content as well as for pH and osmolality before starting the stability study (t0). All samples met the acceptance criteria for formulations according to Table 17 in protein content (165 mg/mL±10% pembrolizumab) and pH (target±0.2).

Osmolality in all samples was detected in a range from 264 mOsmol/kg to 274 mOsmol/kg, values close to isotonic conditions, thereby limiting the sensation of pain during subcutaneous injection.

Osmolality was determined by freezing point depression on an osmometer (Osmomat 030 D-RS from Gonotec). Calibration was performed using a sodium chloride standard (500 mOsmol/kg & 850 mOsmol/kg) and purified water. Samples were measured at a dilution to 50 mg/mL pembrolizumab concentration. Purified water was used for dilution.

The pH was analyzed by the use of a SevenExcellence Multiparameter system and an InLab Micro Pro-ISM pH electrode, both from Mettler Toledo. Measurements were conducted at 23° C.-25° C. in accordance with USP using 150 μl of solution, filled in a 0.5 mL vial. For protein content determination a NanoPhotometer N120 from Implen was used. Here 2 μl of sample solution were diluted with factor 1:10 in their associated placebo buffer solution, quantified at 280 nm, using an extinction coefficient of 1.418 l/g*cm. Background correction was performed using a wavelength of 320 nm.

During the stability program all samples were not altered in their solution pH, all samples met the criteria of 5.5±0.2 (formulations (1), (3), (4), (5)) or 5.2±0.2 (formulation (2)), respectively, under all storage conditions. The pH was determined as single measurement. Both acidic acid as well as adipic acid are buffering well in the defined pH range.

Also, the concentration of pembrolizumab was stable during the complete stability program and all results were quantified within the acceptance criteria 165 mg/mL±10% pembrolizumab. The pembrolizumab concentration was determined in duplicates.

When analyzing the different formulations for their visual appearance, solutions of formulations (1), (2), (3) and (4) appeared as clear, slightly yellowish with a slight opalescence and no visible particles could be detected during storage for 9 months. Identical results were achieved after stressing the samples by repeated freeze/thaw cycles or by mechanical stress.

In contrast, formulation (5), comprising 120 mmol/L trehalose and 70 mM L-lysine, showed the presence of visible particles starting at 3 months storage at 5° C., leading to insoluble precipitates of pembrolizumab. Visible particles were also detected in formulation (5) in samples stored at accelerated conditions.

For visual inspection all 4 vials prepared for each stability condition and pull point were evaluated.

Analyzing all formulations for turbidity using a Nephelostar showed constant values, there was no increase after performing the stability program and stress tests. All values were analyzed in very low range from 5-6 NTU after 9 months storage at 5° C.

Subvisible particles were analyzed using a FlowCam 8100 Multi Objective from Anasysta. These micron-sized protein aggregates and particles are important quality attributes of therapeutic protein formulations due to their risk of enhancing an immunogenic response. The method was adjusted to the following parameters: efficiency of analysis: 60-70%; auto image range: 30 fps; distance to nearest neighbor: 3 μm; flow rate: 0.150 mL/min; processed sample volume: 0.100 mL.

After mechanical stress, freeze/thaw stress as well as after 9 months incubation at 5° C., 3 months at 25° C., 1 month at 40° C. all formulations perform well showing numbers within USP<788> Pharmacopoeia limits. For analyzing subvisible particles by FlowCam all measurements were performed in duplicates.

b. Analysis of High Molecular Weight Species (HMWS) by SE-HPLC

The study samples were diluted in 20 mmol/L L-histidine pH 5.5 to a final concentration of 1 mg/mL and 5 μL thereof were injected to a TSKgel UP-SW3000, (Tosoh, 4.6×150 mm, 2 μm) column to detect high molecular weight species (HMWS) of pembrolizumab.

The protein was eluted by isocratic elution using 0.1 M sodium phosphate buffer with 0.05% (v/v) sodium azide (pH 6.7) at a flow rate of 0.35 mL/min at 25° C. Eluted species were detected at a wavelength of 280 nm and displayed on a graph showing the concentration of the eluted species versus time. The elution profile showed a monomer peak with the non-aggregated protein and peaks of the protein representing higher molecular weight species (HMWS) of the protein. The areas of all peaks were determined.

Table 19 summarizes the percentage of peak area for the HMWS in relation to the total peak area of the eluted species for the formulations shown in Table 17. Each sample was measured as single measurement.

TABLE 19
Overview of HMWS determined using SE-HPLC

| Condition | Formulation | HMWS [%] |
|---|---|---|
| t0 | (1) | 0.5 |
| | (2) | 0.5 |
| | (3) | 0.5 |
| | (4) | 0.6 |
| | (5) | 0.6 |
| 24 h rotation | (1) | 0.7 |
| overhead | (2) | 0.7 |
| | (3) | 0.7 |
| | (4) | 0.7 |
| | (5) | 0.8 |
| 24 h orbital | (1) | 0.7 |
| shaking | (2) | 0.7 |
| | (3) | 0.8 |
| | (4) | 0.8 |
| | (5) | 0.9 |
| F/T | (1) | 0.7 |
| | (2) | 0.7 |
| | (3) | 0.7 |
| | (4) | 0.7 |
| | (5) | 0.8 |
| 2 weeks 40° C. | (1) | 1.4 |
| | (2) | 1.4 |
| | (3) | 1.6 |
| | (4) | 1.7 |
| | (5) | 1.8 |
| 1 month 5° C. | (1) | 0.8 |
| | (2) | 0.8 |
| | (3) | 0.8 |
| | (4) | 0.8 |
| | (5) | 0.9 |
| 1 month 25° C. | (1) | 1.2 |
| | (2) | 1.1 |
| | (3) | 1.2 |
| | (4) | 1.3 |
| | (5) | 1.4 |
| 1 month 40° C. | (1) | 1.7 |
| | (2) | 1.7 |
| | (3) | 1.8 |
| | (4) | 2.1 |
| | (5) | 2.1 |
| 2 months 25° C. | (1) | 1.4 |
| | (2) | 1.3 |
| | (3) | 1.4 |
| | (4) | 1.5 |
| | (5) | 1.6 |
| 3 months 5° C. | (1) | 1.0 |
| | (2) | 0.9 |
| | (3) | 0.9 |
| | (4) | 1.0 |
| | (5) | 1.1 |
| 3 months 25° C. | (1) | 1.5 |
| | (2) | 1.5 |
| | (3) | 1.5 |
| | (4) | 1.7 |
| | (5) | 1.8 |
| 6 months 5° C. | (1) | 1.1 |
| | (2) | 1.0 |
| | (3) | 1.1 |
| | (4) | 1.2 |
| | (5) | 1.2 |
| 9 months 5° C. | (1) | 1.1 |
| | (2) | 1.0 |
| | (3) | 1.1 |
| | (4) | 1.2 |
| | (5) | 1.3 |

After storage for up to 9 months at 5° C. (target storage condition), the HMWS increased only slightly, showing excellent stability. Lowest increases were achieved when using 150 mmol/L L-arginine in formulations (1), (2) and (3), followed by formulation (4) containing lysine. Worse results were achieved, both at target storage condition 2° C.-8° C. as well as at accelerated stability conditions 25° C./40° C., for formulation (5), containing L-lysine and trehalose.

Overall, all formulations containing 150 mmol/L L-arginine showed very promising stabilizing effects on pembrolizumab against the generation of HMWS in both tested buffer systems adipate and acetate, and over a pH range of 5.2 to 5.5. HMWS increased after 9 months storage by A 0.6%, leading to a total HMWS content of 1.0% to 1.1%.

c. Analysis of Colloidal Stability (Kd) by Dynamic Light Scattering

Protein-protein interactions were quantified by kD using a DLS (dynamic light scattering) system from Wyatt Technology (Santa Barbara, California, USA). The kD value is a measure of the balance between repulsive and attractive intermolecular forces. A positive kD value reveals repulsive forces between the proteins in solutions and hence a reduced tendency for aggregation as the surface net charge of the proteins keeps them separated. Negative values for kD are related to attractive net forces between the molecules.

Before analysis all samples were 0.02 μm filtered and diluted with 0.02 μm filtered placebo solution to seven concentrations ranging from 1 mg/mL to 10 mg/mL. For analysis (using Dynamics software, version 8.1.2.144 from Wyatt) the default settings for refractive index and viscosity of water were used. Measurements were performed as duplicate measurements, each with 35 acquisitions of 1 second each as measurement parameter. KD value for each formulation was derived by linear regression using the equation D=D0 (1+kD×c), whereas D=diffusion coefficient (cm2/s), D0=diffusion coefficient (cm2/s) at c=0, kD=diffusion interaction parameter (mL/mg), c=concentration of pembrolizumab.

Samples (1) to (3), all comprising 150 mmol/L L-arginine without the presence of trehalose dihydrate and buffered either by acetate or adipate showed best results, indicated by higher Kd (absolute number of negative values is smaller), compared to other tested formulations. The best formulation identified in this analysis was formulation (2), using 150 mmol/L L-arginine with pH of 5.2, also compared to formulation (1), which is the identical formulation but at pH 5.5. Because of this a lower pH, here around 5.2±0.2 seems to be beneficial to support colloidal stability.

TABLE 20
Kd measurements of highly concentrated
formulations of pembrolizumab

| Formulation | Kd viscosity corrected * 1000 |
|---|---|
| (1) | −7.9 |
| (2) | −5.5 |
| (3) | −8.3 |
| (4) | −9.5 |
| (5) | −11.8 |

d. Detection of Acidic and Basic Species by IEX-HPLC

The study samples were diluted in eluent A (20 mM MES, pH 6.2) to a final concentration of 1 mg/mL and 30 μL thereof were injected onto a MabPac SCX-10 (Thermo Scientific, 4×250 mm, 10 μm) column in order to detect modifications of pembrolizumab leading to charge heterogeneities. Proteins were eluted using a mixture of mobile phase A (20 mM MES, pH 6.2) and B (20 mM MES, 120 mM KCl, pH 6.2) at a flow rate of 1 mL/min at 40° C. by applying a salt gradient (KCl). Eluted species were detected at a wavelength of 280 nm and displayed on a graph showing the concentration of the eluted species versus time. The elution profile showed a main peak as well as several peaks representing the acidic and basic variants. The areas of all peaks were determined. Table 21 summarizes the percentage of the peak area for the acidic species in relation to the total peak area of the eluted species for the formulations shown in Table 17. Each sample was measured in triplicates.

TABLE 21
Overview of acidic and basic species determined using IEX-HPLC

| | | Acidic | Main | Basic |
|---|---|---|---|---|
| Condition | Formulation | species [%] | peak [%] | species [%] |
| t0 | (1) | 20.1 | 54.0 | 25.9 |
| | (2) | 20.2 | 54.1 | 25.7 |
| | (3) | 20.2 | 54.2 | 25.6 |
| | (4) | 20.2 | 54.1 | 25.6 |
| | (5) | 20.2 | 54.2 | 25.6 |
| 24 h rotation | (1) | 20.2 | 54.2 | 25.6 |
| overhead | (2) | 20.2 | 54.3 | 25.5 |
| | (3) | 20.2 | 54.3 | 25.4 |
| | (4) | 20.3 | 54.2 | 25.5 |
| | (5) | 20.3 | 54.3 | 25.5 |
| 24 h orbital | (1) | 20.2 | 54.0 | 25.8 |
| shaking | (2) | 20.2 | 54.1 | 25.7 |
| | (3) | 20.3 | 54.2 | 25.5 |
| | (4) | 20.2 | 54.2 | 25.6 |
| | (5) | 20.3 | 54.3 | 25.5 |
| F/T | (1) | 20.1 | 54.1 | 25.8 |
| | (2) | 20.2 | 54.2 | 25.7 |
| | (3) | 20.2 | 54.3 | 25.5 |
| | (4) | 20.1 | 55.0 | 25.0 |
| | (5) | 20.2 | 54.2 | 25.6 |
| 2 weeks 40° C. | (1) | 26.3 | 48.9 | 24.9 |
| | (2) | 25.7 | 49.5 | 24.8 |
| | (3) | 26.6 | 49.7 | 23.7 |
| | (4) | 26.4 | 49.1 | 24.4 |
| | (5) | 26.9 | 49.4 | 23.7 |
| 1 month 5° C. | (1) | 20.1 | 54.3 | 25.6 |
| | (2) | 20.2 | 54.2 | 25.6 |
| | (3) | 20.2 | 54.4 | 25.4 |
| | (4) | 20.2 | 54.3 | 25.5 |
| | (5) | 20.2 | 54.3 | 25.4 |
| 1 month 25° C. | (1) | 20.5 | 53.9 | 25.6 |
| | (2) | 20.6 | 53.1 | 25.4 |
| | (3) | 20.7 | 54.3 | 25.0 |
| | (4) | 20.6 | 54.1 | 25.2 |
| | (5) | 20.7 | 54.3 | 24.9 |
| 1 month 40° C. | (1) | 32.7 | 44.6 | 22.7 |
| | (2) | 32.1 | 44.5 | 23.4 |
| | (3) | 33.4 | 44.9 | 21.7 |
| | (4) | 33.5 | 44.0 | 22.5 |
| | (5) | 34.2 | 44.0 | 21.7 |
| 2 months 25° C. | (1) | 21.2 | 53.2 | 25.6 |
| | (2) | 21.1 | 53.6 | 25.3 |
| | (3) | 21.4 | 53.8 | 24.8 |
| | (4) | 21.2 | 53.5 | 25.3 |
| | (5) | 21.4 | 54.0 | 24.6 |
| 3 months 5° C. | (1) | 20.0 | 54.1 | 26.0 |
| | (2) | 20.0 | 54.4 | 25.7 |
| | (3) | 20.0 | 54.5 | 25.4 |
| | (4) | 20.1 | 54.3 | 25.6 |
| | (5) | 20.1 | 54.4 | 25.5 |
| 3 months 25° C. | (1) | 22.0 | 53.3 | 24.7 |
| | (2) | 21.7 | 53.2 | 25.0 |
| | (3) | 22.2 | 53.7 | 24.1 |
| | (4) | 22.0 | 53.5 | 24.5 |
| | (5) | 22.2 | 53.9 | 23.9 |
| 6 months 5° C. | (1) | 19.7 | 53.7 | 26.5 |
| | (2) | 19.8 | 54.0 | 26.2 |
| | (3) | 20.0 | 54.1 | 25.8 |
| | (4) | 19.9 | 54.0 | 26.0 |
| | (5) | 20.1 | 54.1 | 25.9 |
| 9 months 5° C. | (1) | 19.6 | 54.4 | 26.0 |
| | (2) | 19.8 | 54.5 | 25.7 |
| | (3) | 19.9 | 54.6 | 25.5 |
| | (4) | 19.7 | 54.5 | 25.7 |
| | (5) | 20.1 | 54.5 | 25.5 |

After storage for up to 9 months at 5° C. (target storage condition), both the acidic species and basic species remained near t0 level for all formulations tested within this stability study, promising long term stability. Acidic species decreased after 9 months at 5° C. by A 0.1% to 0.5%, basic species increased/decreased by A 0.1% main peak was determined with A 0.3% to 0.4%, minimal variations to the starting conditions.

e. Detection of Acidic and Basic Species by icIEF

The aim of imaged capillary isoelectric focusing (icIEF) is to determine the isoelectric point (pl) and the heterogeneity of charge isoforms of a protein that are caused by posttranslational modifications (PTM). The power of icIEF lies in the high resolving electropherograms allowing a precise and reproducible relative quantification of the charge isoforms.

Samples were rebuffered by ultrafiltration against ultrapure water and interfering buffer components were depleted from the samples using Detergent Removal Spin Columns (Pierce) according to the manufacturer's instructions. The protein concentration was determined by UV measurement (Absorption at 280 nm). The rebuffered sample was diluted to 1 g/L with ultrapure water. 40 μg diluted sample (corresponding to a final protein concentration of 0.2 g/L) were mixed with 2% ampholytes (0.25% pH 9-11 and 3% 8-10.5), 0.35% methylcellulose, 4 M Urea and pl markers (8.18 and 9.99). Focusing was performed in two steps (1 min at 1500 V and 10 min at 3000 V).

Final analysis was carried out with the imaged CEsystem Maurice C (ProteinSimple). Data were evaluated by pl calibration of the electropherograms using the two internal pl markers and Compass for iCE software (ProteinSimple). Peak integration was performed using Empower software (Waters). Calculations with relative peak areas (e.g. for peak grouping) were performed using initial values for relative peak areas with three decimal places. The initial values used for calculations and the calculated results are reported rounded to one decimal place. Samples shown in Table 17 were analyzed as single measurement after incubation for 3 months at 40° C./75% relative humidity, for 6 months at 25° C./60% relative humidity and for 9 months at 5° C.

TABLE 22
Overview of acidic and basic species determined using clEF

| | | Acidic | Main | Basic |
|---|---|---|---|---|
| Condition | Formulation | species [%] | Peak [%] | species [%] |
| t0 | (1) | 23.2 | 54.1 | 22.6 |
| | (2) | 23.1 | 53.8 | 23.1 |
| | (3) | 23.4 | 54.1 | 22.5 |
| | (4) | 23.8 | 53.7 | 22.4 |
| | (5) | 23.2 | 54.0 | 22.7 |
| 24 h rotation | (1) | 24.2 | 53.3 | 22.5 |
| overhead | (2) | 22.9 | 54.2 | 23.0 |
| | (3) | 23.5 | 53.9 | 22.6 |
| | (4) | 23.4 | 53.9 | 22.7 |
| | (5) | 23.2 | 54.3 | 22.5 |
| 24 h orbital | (1) | 24.4 | 53.0 | 22.6 |
| shaking | (2) | 24.6 | 53.1 | 22.3 |
| | (3) | 24.8 | 52.9 | 22.4 |
| | (4) | 25.1 | 52.7 | 22.2 |
| | (5) | 25.0 | 52.7 | 22.3 |
| F/T | (1) | 24.5 | 52.3 | 22.3 |
| | (2) | 25.2 | 52.2 | 22.6 |
| | (3) | 25.5 | 52.1 | 22.4 |
| | (4) | 25.3 | 52.6 | 22.1 |
| | (5) | 25.2 | 52.5 | 22.3 |
| 2 weeks 40° C. | (1) | 28.0 | 50.3 | 21.6 |
| | (2) | 27.1 | 51.0 | 21.9 |
| | (3) | 28.1 | 50.5 | 21.3 |
| | (4) | 27.6 | 51.0 | 21.4 |
| | (5) | 28.1 | 50.9 | 21.0 |
| 1 month 5° C. | (1) | 22.3 | 54.7 | 23.1 |
| | (2) | 22.7 | 54.4 | 22.9 |
| | (3) | 22.1 | 54.8 | 23.1 |
| | (4) | 23.0 | 53.8 | 23.2 |
| | (5) | 22.8 | 54.5 | 22.8 |
| 1 month 25° C. | (1) | 22.9 | 54.2 | 23.0 |
| | (2) | 23.2 | 53.7 | 23.1 |
| | (3) | 23.3 | 53.9 | 22.8 |
| | (4) | 23.4 | 53.8 | 22.8 |
| | (5) | 23.5 | 54.2 | 22.3 |
| 1 month 40° C. | (1) | 34.0 | 46.6 | 19.5 |
| | (2) | 31.7 | 47.6 | 20.7 |
| | (3) | 33.9 | 47.0 | 19.1 |
| | (4) | 33.6 | 46.8 | 19.5 |
| | (5) | 33.8 | 47.4 | 18.8 |
| 2 months 25° C. | (1) | 25.5 | 52.3 | 22.3 |
| | (2) | 25.3 | 52.4 | 22.3 |
| | (3) | 25.7 | 52.2 | 22.1 |
| | (4) | 25.9 | 52.0 | 22.1 |
| | (5) | 26.2 | 51.9 | 21.8 |
| 3 months 5° C. | (1) | 23.4 | 53.8 | 22.8 |
| | (2) | 24.7 | 53.1 | 22.3 |
| | (3) | 25.0 | 52.3 | 22.6 |
| | (4) | 24.9 | 52.9 | 22.2 |
| | (5) | 25.0 | 52.3 | 22.6 |
| 3 months 25° C. | (1) | 27.1 | 51.3 | 21.6 |
| | (2) | 27.1 | 51.1 | 21.7 |
| | (3) | 27.5 | 51.3 | 21.2 |
| | (4) | 27.3 | 51.0 | 21.7 |
| | (5) | 27.6 | 50.8 | 21.6 |
| 6 months 5° C. | (1) | 23.2 | 53.8 | 23.0 |
| | (2) | 23.5 | 53.9 | 22.6 |
| | (3) | 23.3 | 54.0 | 22.7 |
| | (4) | 23.4 | 53.8 | 22.8 |
| | (5) | 24.1 | 53.7 | 22.3 |
| 9 months 5° C. | (1) | 26.2 | 51.7 | 22.1 |
| | (2) | 26.8 | 51.1 | 22.1 |
| | (3) | 26.5 | 52.1 | 21.4 |
| | (4) | 26.4 | 51.1 | 22.5 |
| | (5) | 26.2 | 51.7 | 22.1 |

f. Analysis of Low Molecular Weight Species (LMWS) by Non-Reducing cGE (LabChip)

Non-reducing capillary gel electrophoresis (cGE) was performed to quantify LMWS in pembrolizumab formulations, using a Perkin Elmer Labchip GXII Touch Protein Characterization System, a HT Protein Express Chip (760499, Perkin Elmer) and Protein Express assay reagent kit (CLS960008, Perkin Elmer). The samples were prepared according to the manufacturer's general instructions as described in the Kit handbook (PN CLS140156, Rev. D) and in the following: Formulated pembrolizumab samples were diluted with water to a concentration of 1 mg/mL and 10 μl thereof were further diluted with 35 μl of denaturation buffer. The denaturation buffer was composed of sample buffer (from reagent kit), LDS (lithium dodecyl sulfate, L4632, from Sigma Aldrich) and NEM (N-Ethylmaleimide, E1271, from Sigma Aldrich). Non-reducing conditions were established with stocks of 200 mmol/L NEM added at 1:20 v/v ratio. To achieve a more homogeneous sample denaturation, the denaturation solution was supplemented with LDS at a concentration of 1% (v/v). Sample denaturation was performed at 75° C. for 10 min in a Bio-Rad real-time PCR cycler. Denatured samples were stored overnight at room temperature and protected from light, the assay concentration was established by adding 35 μl water (from B. Braun) directly prior running the assay.

The samples were analyzed in triplicate wells. Electrophoretic separation was performed using the LabChip GXII Touch HT software, running the preset P200 Antibody Analysis (Perkin Elmer). Electropherogram profiles as well as purity (main peak) and impurity (LMWS, HMWS) content were monitored.

TABLE 23
Overview of LMWS determined using SDS-cGE (non-red.)

| Condition | Formulation | LMWS [%] |
|---|---|---|
| to | (1) | 0.5 |
| | (2) | 0.5 |
| | (3) | 0.5 |
| | (4) | 0.5 |
| | (5) | 0.5 |
| 24 h rotation | (1) | 0.5 |
| overhead | (2) | 0.5 |
| | (3) | 0.5 |
| | (4) | 0.5 |
| | (5) | 0.6 |
| 24 h orbital | (1) | 0.5 |
| shaking | (2) | 0.5 |
| | (3) | 0.5 |
| | (4) | 0.5 |
| | (5) | 0.5 |
| F/T | (1) | 0.5 |
| | (2) | 0.5 |
| | (3) | 0.5 |
| | (4) | 0.5 |
| | (5) | 0.5 |
| 2 weeks 40° C. | (1) | 0.9 |
| | (2) | 0.9 |
| | (3) | 0.9 |
| | (4) | 0.9 |
| | (5) | 0.8 |
| 1 month 5° C. | (1) | 0.7 |
| | (2) | 0.6 |
| | (3) | 0.7 |
| | (4) | 0.6 |
| | (5) | 0.6 |
| 1 month 25° C. | (1) | 0.7 |
| | (2) | 0.7 |
| | (3) | 0.7 |
| | (4) | 0.7 |
| | (5) | 0.7 |
| 1 month 40° C. | (1) | 1.4 |
| | (2) | 1.4 |
| | (3) | 1.5 |
| | (4) | N/A |
| | (5) | 1.4 |
| 2 months 25° C. | (1) | 1.0 |
| | (2) | 0.9 |
| | (3) | 1.0 |
| | (4) | 0.9 |
| | (5) | 0.9 |
| 3 months 5° C. | (1) | 0.5 |
| | (2) | 0.5 |
| | (3) | 0.5 |
| | (4) | 0.5 |
| | (5) | 0.5 |
| 3 months 25° C. | (1) | 0.8 |
| | (2) | 0.8 |
| | (3) | 0.8 |
| | (4) | 0.7 |
| | (5) | 0.7 |
| 6 months 5° C. | (1) | 0.7 |
| | (2) | 0.6 |
| | (3) | 0.6 |
| | (4) | 0.6 |
| | (5) | 0.6 |
| 9 months 5° C. | (1) | 0.4 |
| | (2) | 0.4 |
| | (3) | 0.4 |
| | (4) | 0.4 |
| | (5) | 0.4 |

After storage for up to 9 months at 500 (target storage condition), LMWS were measured in a comparable range to t0 values, no increase was quantified in all formulations tested within this stability study, promising successful long term storage stability with regard to the generation of LMWS. In addition, all formulations remained stable after multiple freeze/thaw cycles and after applying mechanical stress. Overall, all formulations tested within this study showed similar and very good results.

q. Detection of Oxidized Species by HIC

Hydrophobic interaction chromatography (HIC) was used to assess oxidation of pembrolizumab. HIC is based on the interaction between the hydrophobic ligands on the stationary phase (HIC column) and the hydrophobic surface of proteins, impurities can be separated due to differences in binding to the stationary phase.

The study samples were diluted in dilution buffer (10 mM L-histidine, 7% (w/v) sucrose, 0.02% polysorbate 80, 200 mM L-methionine, pH 5.5) to a final concentration of 1 mg/mL. For each 125 μg sample 1 μg carboxypeptidase B (CPB) was added, mixed and incubated 60 min at 25° C. using a thermal shaker with cooling function set to 500 rpm. 5 μl (5 μg) thereof were injected onto a TSKgel Butyl-NPR (TOSOH, 4.6×35 mm, 2.5 μm) column in order to detect oxidations of pembrolizumab. Proteins were eluted using a mixture of mobile phase A (2 M ammonium sulfate in 100 mM sodium phosphate buffer, pH 7.0) and B (100 mM sodium phosphate buffer, pH 7.0) at a flow rate of 0.8 mL/min at 30° C. by using a ACQUITY UPLC H-Class Bio system from Waters with applying a ammonium salt gradient according to Table 24.

TABLE 24
Gradient for HIC analysis

| Time [min] | % A | % B |
|---|---|---|
| Initial | 87 | 13 |
| 1.00 | 80 | 20 |
| 26.00 | 55 | 45 |
| 36.00 | 0 | 100 |
| 37.00 | 0 | 100 |

Eluted species were detected using a fluorescence detector with a wavelength of 278 nm for excitation and 350 nm for emission. Elution profile was displayed on a graph showing the concentration of the eluted species versus time. The areas of peak 1 and peak 2 showing oxidized variants were determined and calculated in relation to the total peak area of the eluted species for the formulations shown in Fehler!Verweisquelle konnte nicht gefunden werden.7. Each sample was analyzed in duplicate, all results are shown in Table 25.

TABLE 25
Overview of oxidation levels of pembrolizumab (oxidation
peak 01, oxidation peak 02, sum of oxidation peak
01 and oxidation peak 02) determined using HIC

| | | Oxidation | Oxidation | |
|---|---|---|---|---|
| | | Peak | peak | Sum |
| Condition | Formulation | 01 [%] | 02 [%] | oxidation [%] |
| t0 | (1) | 0.32 | 7.44 | 7.76 |
| | (2) | 0.31 | 7.41 | 7.72 |
| | (3) | 0.30 | 7.36 | 7.66 |
| | (4) | 0.35 | 7.35 | 7.70 |
| | (5) | 0.36 | 7.45 | 7.81 |
| 24 h rotation | (1) | 0.33 | 7.50 | 7.83 |
| overhead | (2) | 0.34 | 7.43 | 7.77 |
| | (3) | 0.36 | 7.40 | 7.76 |
| | (4) | 0.29 | 7.37 | 7.66 |
| | (5) | 0.35 | 7.44 | 7.79 |
| 24 h orbital | (1) | 0.31 | 7.46 | 7.77 |
| shaking | (2) | 0.31 | 7.45 | 7.76 |
| | (3) | 0.36 | 7.45 | 7.81 |
| | (4) | 0.36 | 7.44 | 7.80 |
| | (5) | 0.36 | 7.45 | 7.81 |
| F/T | (1) | 0.33 | 7.45 | 7.78 |
| | (2) | 0.30 | 7.37 | 7.67 |
| | (3) | 0.34 | 7.34 | 7.68 |
| | (4) | 0.33 | 7.36 | 7.69 |
| | (5) | 0.32 | 7.43 | 7.75 |
| 2 weeks 40° C. | (1) | 0.60 | 9.49 | 10.09 |
| | (2) | 0.59 | 9.15 | 9.74 |
| | (3) | 0.56 | 9.40 | 9.96 |
| | (4) | 0.53 | 9.31 | 9.84 |
| | (5) | 0.54 | 9.19 | 9.73 |
| 1 month 5° C. | (1) | 0.36 | 7.71 | 8.07 |
| | (2) | 0.31 | 7.57 | 7.88 |
| | (3) | 0.30 | 7.59 | 7.89 |
| | (4) | 0.38 | 7.61 | 7.99 |
| | (5) | 0.34 | 7.60 | 7.94 |
| 1 month 25° C. | (1) | 0.43 | 8.50 | 8.93 |
| | (2) | 0.37 | 8.29 | 8.66 |
| | (3) | 0.37 | 8.39 | 8.76 |
| | (4) | 0.41 | 8.43 | 8.84 |
| | (5) | 0.42 | 8.40 | 8.82 |
| 1 month 40° C. | (1) | 0.75 | 11.09 | 11.84 |
| | (2) | 0.74 | 10.68 | 11.42 |
| | (3) | 0.74 | 11.18 | 11.92 |
| | (4) | 0.66 | 10.98 | 11.64 |
| | (5) | 0.60 | 10.68 | 11.28 |
| 2 months 25° C. | (1) | 0.55 | 9.30 | 9.85 |
| | (2) | 0.73 | 9.08 | 9.81 |
| | (3) | 0.62 | 9.21 | 9.83 |
| | (4) | 0.68 | 9.15 | 9.83 |
| | (5) | 0.72 | 9.01 | 9.73 |
| 3 months 5° C. | (1) | 0.29 | 7.77 | 8.06 |
| | (2) | 0.28 | 7.60 | 7.88 |
| | (3) | 0.35 | 7.71 | 8.06 |
| | (4) | 0.29 | 7.60 | 7.89 |
| | (5) | 0.33 | 7.67 | 8.00 |
| 3 months 25° C. | (1) | 0.46 | 9.59 | 10.05 |
| | (2) | 0.45 | 9.35 | 9.80 |
| | (3) | 0.47 | 9.59 | 10.06 |
| | (4) | 0.47 | 9.50 | 9.97 |
| | (5) | 0.44 | 9.28 | 9.72 |
| 6 months 5° C. | (1) | 0.35 | 7.76 | 8.11 |
| | (2) | 0.32 | 7.76 | 8.08 |
| | (3) | 0.33 | 7.78 | 8.11 |
| | (4) | 0.34 | 7.80 | 8.14 |
| | (5) | 0.31 | 7.77 | 8.08 |
| 9 months 5° C. | (1) | 0.33 | 7.99 | 8.32 |
| | (2) | 0.33 | 7.83 | 8.16 |
| | (3) | 0.33 | 7.91 | 8.24 |
| | (4) | 0.33 | 7.89 | 8.22 |
| | (5) | 0.34 | 7.88 | 8.22 |

After storage for up to 9 months at 5° C. (target storage condition), oxidation levels just slightly increased to a total level in the range about 8.2% to 8.3 compared to t0 (increase of A 0.4% to 0.7%) for all formulations tested within this stability study, promising successful long term storage stability with regard to oxidation of pembrolizumab. Oxidation peak 01 remained stable in all formulations after 9 months storage at 5° C.

In addition, all formulations remained stable after multiple freeze/thaw cycles and after applying mechanical stress. Overall, all formulations tested within this study showed similar very good results.

h. Analysis of Shear Viscosity

Shear viscosity was determined at 20° C. by rheometry with a cone-plated geometry using a Kinexus ultra plus rheometer (diameter: 40 mm, cone angle: 0.5°; sample volume 160 μl; CP 0.5°/40 mm). To avoid evaporation, a solvent trap filled with water was used. Calibration was carried out with known viscosity prior to every sequence. The samples were equilibrated for 2 minutes prior to each measurement. After ramping up from 0.1 to 1000 s⁻¹, the dynamic viscosity was measured for one minute (one datapoint per second) at constant shear rate of 1000 s⁻¹. All measurements were performed as single measurement.

All formulations prepared according to Table 17 were analyzed for their shear viscosity after preparation, results are shown in Table 26 Table.

It was demonstrated that formulations containing L-arginine effectively reduced shear viscosity in formulations (1), (2) and (3) to very low levels when compared to the lysine-containing formulations (4) and (5).

Nevertheless, the values for all formulations are well within the tolerance limits of subcutaneous injections.

TABLE 26
Viscosity measurement of high concentration pembrolizumab

| Formulation | Shear viscosity [mPa*sec) |
|---|---|
| (1) | 10.0 |
| (2) | 12.9 |
| (3) | 14.0 |
| (4) | 20.5 |
| (5) | 20.0 |

i. Summary of Study Results

Based on the results obtained in this stability program novel compositions could be identified which can stabilize highly concentrated pembrolizumab in liquid formulations.

Especially the use of 150 mmol/L L-arginine improved stability of pembrolizumab at 165 mg/mL in a pH range of 5.2 to 5.5 (formulations (1) (2) and (3)), showing pH robustness of this stabilizing effect when comparing formulation (1) and (2). Two buffer systems providing buffer capacity at the tested pH were evaluated, leading to very comparable results for 10 mmol/L acetate and 10 mmol/L adipate buffer system (formulations (1) and (3)).

These three formulations—either adipate or acetate buffered—worked very well in stabilizing high concentrations of pembrolizumab. Comparably good results were also achieved when using 140 mmol/L L-lysine in combination with 10 mmol acetic acid as buffering system (formulation (4)), showing general stabilizing effects of basic amino acids (in this study represented by L-arginine and L-lysine) in combination with pembrolizumab. Nevertheless, using L-arginine showed additionally improvements in lowering the viscosity of the solution.

The combination of L-lysine with trehalose led to visible particles after 3 months, also the colloidal stability (Kd) analyzed by dynamic light scattering showed a higher level of attractive forces between the molecules, which finally might promote pembrolizumab aggregation during shelf life of formulations of high concentrated pembrolizumab. This was confirmed by storing this formulation at accelerated conditions, leading to increased contents of HMWS analyzed by SE-HPLC.

1. A liquid pharmaceutical composition comprising:
(a) an anti-human PD1 antibody, wherein the anti-human PD1 antibody is pembrolizumab present in a concentration of 100 to 200 mg/ml;
(b) arginine;
(c) a non-ionic surfactant, wherein the non-ionic surfactant is polysorbate 20 or polysorbate 80; and
(d) a mono- or dicarboxylic acid with a backbone of 2-6 carbon atoms or at least one salt thereof, wherein the mono- or dicarboxylic acid with a backbone of 2-6 carbon atoms is acetate or adipate,
wherein the pH of the composition is between 5.0 and 5.8.

2. The liquid pharmaceutical composition of claim 1, wherein the buffer is present in a concentration of 1 mM to 50 mM.

3. The liquid pharmaceutical composition of claim 2, wherein the buffer is present in a concentration of 10 mM.

4. The liquid pharmaceutical composition of claim 1, wherein the non-ionic surfactant is polysorbate 80.

5. The liquid pharmaceutical composition of claim 1, wherein the non-ionic surfactant is present in a concentration of 0.1 mg/ml to 0.4 mg/ml.

6. The liquid pharmaceutical composition of claim 5, wherein the non-ionic surfactant is present in a concentration of 0.2 mg/ml.

7. The liquid pharmaceutical composition of claim 1, comprising arginine in a concentration of 50 mM to 350 mM.

8. The liquid pharmaceutical composition of claim 7, comprising arginine in a concentration of 150 mM.

9. The liquid pharmaceutical composition of claim 1, wherein the pharmaceutical composition does not comprise trehalose.

10. The liquid pharmaceutical composition of claim 9, wherein the pharmaceutical composition does not comprise any sugar or sugar alcohol.

11. The liquid pharmaceutical composition of claim 1, wherein the pharmaceutical composition does not comprise methionine or histidine.

12. The liquid pharmaceutical composition claim 11, wherein the pharmaceutical composition does not comprise any antioxidant.

13. The liquid pharmaceutical composition of claim 1, wherein the anti-human PD1 antibody is present in a concentration of 165 mg/ml or in a concentration of 20 to 80 mg/ml.

14. The liquid pharmaceutical composition of claim 13, wherein the anti-human PD1 antibody is present in a concentration of 25 mg/ml.

15. A liquid pharmaceutical composition consisting of 10-mM-acetate buffer or adipate buffer, arginine or lysine, polysorbate 20 or polysorbate 80, pembrolizumab and water for injection and having a pH of 5.0 to 5.8.

16. The liquid pharmaceutical composition of claim 15, consisting of 10 mM acetate buffer, 150 mM L-arginine, 0.2 mg/ml polysorbate 80, 165 mg/ml and water for injection and having a pH of 5.2 or 5.5.

17. The liquid pharmaceutical composition of claim 15, consisting of 10 mM adipate buffer, 150 mM L-arginine, 0.2 mg/ml polysorbate 80, 165 mg/ml pembrolizumab and water for injection and having a pH of 5.5.

18. The liquid pharmaceutical composition of claim 15, consisting of 10 mM acetate buffer, 140 mM L-lysine, 0.2 mg/ml polysorbate 80, 165 mg/ml pembrolizumab and water for injection and having a pH of 5.5.

19. A method for treatment of cancer, comprising administering the liquid pharmaceutical composition of claim 1.

20. The method for treatment of cancer according to claim 19, wherein the cancer is melanoma, non-small cell lung carcinoma, classical Hodgkin lymphoma, urothelial carcinoma, head and neck squamous cell carcinoma, renal cell carcinoma, colorectal cancer, small cell lung cancer, microsatellite instability-high or mismatch repair deficient cancer, primary mediastinal large B-cell lymphoma, gastric or gastroesophageal junction adenocarcinoma, oesophageal carcinoma, hepatocellular carcinoma, Merkel cell carcinoma, endometrial carcinoma, tumor mutational burden-high cancer, cutaneous squamous cell carcinoma, triple-negative breast cancer or cervical cancer.


SEQUENCE LISTING (10 sequences)

SEQ ID NO: 1 - protein, 15 aa, synthetic construct
  REGION 1..15: note = Pembrolizumab-Light chain CDR1
RASKGVSTSGYSYLH

SEQ ID NO: 2 - protein, 7 aa, synthetic construct
  REGION 1..7: note = Pembrolizumab-Light chain CDR2
LASYLES

SEQ ID NO: 3 - protein, 9 aa, synthetic construct
  REGION 1..9: note = Pembrolizumab-Light chain CDR3
QHSRDLPLT

SEQ ID NO: 4 - protein, 111 aa, synthetic construct [sequence omitted]

SEQ ID NO: 5 - protein, 218 aa, synthetic construct
  REGION 1..218: note = Pembrolizumab-Light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, synthetic construct
  REGION 1..5: note = Pembrolizumab-Heavy chain CDR1
NYYMY

SEQ ID NO: 7 - protein, 17 aa, synthetic construct
  REGION 1..17: note = Pembrolizumab-Heavy chain CDR2
GINPSNGGTNFNEKFKN

SEQ ID NO: 8 - protein, 11 aa, synthetic construct
  REGION 1..11: note = Pembrolizumab-Heavy chain CDR3
RDYRFDMGFDY

SEQ ID NO: 9 - protein, 120 aa, synthetic construct [sequence omitted]

SEQ ID NO: 10 - protein, 447 aa, synthetic construct
  REGION 1..447: note = Pembrolizumab-Heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK